(19) United States
(12) Patent Application Publication
(10) Pub. No.: US 2020/0148777 A1
(43) Pub. Date: May 14, 2020

(54) CANCER-ASSOCIATED IMMUNOSUPPRESSION INHIBITOR

(71) Applicants: GAMAMABS PHARMA, Toulouse (FR); INSTITUT NATIONAL DE LA SANTÉ ET DE LA RECHERCHE MÉDICALE (INSERM), Paris (FR); CENTRE NATIONAL DE LA RECHERCHE SCIENTIFIQUE (CNRS), Paris (FR); UNIVERSITE PARIS DESCARTES, Paris (FR)

(72) Inventors: Jean-Marc BARRET, Castres (FR); Jean-François PROST, Versailles (FR); Mehdi LAHMAR, Viroflay (FR); Stéphane DEGOVE, Pantin (FR); Houcine BOUGHERARA, Cachan (FR); Emmanuel DONNADIEU, Boulogne (FR)

(21) Appl. No.: 16/617,136

(22) PCT Filed: May 29, 2018
(86) PCT No.: PCT/EP2018/064081

§ 371 Date: Nov. 26, 2019

(30) Foreign Application Priority Data
May 29, 2017 (EP) 17305619.3

(51) Int. Cl. C07K 16/28; A61K 39/395

(52) U.S. Cl. CPC C07K 16/2869; A61K 39/3955; C07K 2317/732; C07K 2317/24; C07K 2317/92; C07K 2317/41

(57) ABSTRACT

The present invention relates to a glyco-engineered Fc fragment-bearing compound for its use as an immunosuppression inhibitor in the treatment of a cancer-associated immunosuppression. The invention further relates to a pharmaceutical composition comprising at least this glyco-engineered Fc fragment-bearing compound.

### FIELD OF THE INVENTION

This invention relates to the treatment of an immunosuppression state that may occur during a cancer disease.

### BACKGROUND OF THE INVENTION

Cancer immune evasion is a major strumbling block in designing effective anticancer therapeutic strategies. Although considerable progress has been made in understanding how cancers evade destructive immunity, measures to counteract tumor escape have not kept pace.

Long-term survival of the patient is considered the “gold standard” of success in cancer treatment although, from the patient's perspective, disease-free survival is the ultimate goal. It is increasingly believed that long-term survival and disease-free survival are largely dependent upon enhancing the patient's own immune system to mount an effective antitumor response. Evidence of a strong immune response to therapy, even to the point of inducing autoimmune symptoms, may be a positive indicator of long-term survival in cancer patients (Burkholder et al., 2014, Biochimica et Biophysica Acta, Vol. 1845: 182-201).

Although a variety of agents have been screened for their anti-tumor effects and a selection thereof have been approved for the treatment of cancer patients, chemotherapy, radiation therapy, and surgery remain the mainstays o standard cancer therapeutic strategies (Vinay et al., 2015, Seminars in cancer biology, Vol. 35: 5185-5198). A downside of these therapies is their ability to cause a transient immune suppression which in turn increases the risk of infection and is also likely to decrease the immune system's ability to inhibit further development of cancer.

Identifying adapted global cancer treatment strategies encompasses determining the extent to which immune-boosting therapies may augment standard anti-cancer therapies. It is strongly suggested in the art that most, if not all, global cancer treatment strategies should include associated means that increase antitumor immunity, regardless the kind of antitumor treatment which is used.

Immunotherapies have potential for the treatment of cancer, because immune-based therapies act through a mechanism that is distinct from chemotherapy or radiation therapy and because they represent non-cross-resistant treatments, with an entirely different spectrum of toxicities. Both T and B cells are capable of recognizing a diverse array of potential tumor antigens through the genetic recombination of their respective receptors, and, more importantly, both T and B cells can distinguish small antigenic differences between normal and transformed cells, providing specificity while minimizing toxicity

The importance of an immune response to cancer has been known for decades. However, recent advances in immuno-oncology have greatly improved the understanding of the immune system and cancer interactions. Immunoediting refers to the process where the immune system can alter tumor progression. It regulates both tumor quantity and quality.

The process of cancer immunoediting has three distinct phases: elimination, equilibrium and escape phase, respectively. The escape phase may occur at the tumor level or at the level of the tumor microenvironment. At the microenvironment level, the recruitment of regulatory T cells (Tregs) and myeloid derived suppressor cells (MDSCs) or expression of programmed death-1 (PD-1)/programmed death-ligand 1 (PD-L1) in immune infiltrates may lead to an immunosuppressive tumor microenvironment.

Tumor-associated macrophages (TAMs), tumor-associated fibroblasts, Tregs, and soluble factors produced by suppressor cells all contribute to cancer-induced immune suppression. TAMs may drive multiple protumor processes, including immunosuppression, angiogenesis, and secretion of direct tumor growth factors.

Thus, the immune system plays an important role in controlling and eradicating cancer. Nevertheless, in the setting of malignancy, multiple mechanisms of immune suppression may exist that prevent effective antitumor immunity.

Antibody therapy directed against several negative immunologic regulators (checkpoints) has today demonstrated significant success and is likely to be a major component of treatment for patients with a variety of malignancies.

The first of such molecules shown to inhibit both T-cell proliferation and IL-2 production was cytotoxic T-lymphocyte associated protein 4 (CTLA-4). With this discovery, efforts turned to blocking this inhibitory pathway in an attempt to activate dormant T-cells directed at cancer cells. The first antibody directed against CTLA-4, ipilimumab, was quickly ushered into clinical trials and was approved by the US Food and Drug Administration (FDA) for the treatment of metastatic melanoma in 2011. Following the success of ipilimumab, other immune checkpoints were studied as possible targets for inhibition. One such interaction was that of the programmed cell death-1 (PD-1) T-cell receptor and its ligand found on many cancer cells, programmed death-ligand 1 (PD-L1).

However, those antibodies are efficacious in limited types of tumors (mainly melanoma, lung cancers, kidney cancers) and, even in the sensitive tumors, an important proportion of patients remain resistant.

It flows from the presently acquired knowledge relating to anti-cancer therapeutic strategies that, in most situations, dual approaches shall be followed which seek to (i) eliminate immune suppressing factors/mechanisms, and (ii) enhance tumor-killing activities, so as to achieve successful cancer therapy.

Thus, there is still a need in the art to provide further therapeutic strategies for treating cancers. There is especially a need for novel means for reducing or blocking immunosuppression that may occur in cancer patients, so as to define novel successful anti-cancer treatment strategies.

### SUMMARY OF THE INVENTION

The present invention relates to a glyco-engineered Fc fragment-bearing compound for its use as an immunosuppression inhibitor in the treatment of a cancer-associated immunosuppression.

Notably, the present invention relates to a glyco-engineered Fc fragment-bearing compound for its use as a T-cell immunosuppression inhibitor in the treatment of a cancer-associated immunosuppression.

This invention encompasses a glyco-engineered Fc fragment-bearing compound for its use as a CD8⁺ T-cell immunosuppression inhibitor in the treatment of a cancer-associated immunosuppression.

In some embodiments, the said glyco-engineered Fc fragment-bearing compound is a hypofucosylated Fc fragment-bearing compound.

In some embodiments, the glyco-engineered Fc fragment-bearing compound comprises two amino acid chains of SEQ ID NO. 70

In some embodiments, the said glyco-engineered Fc fragment-bearing compound is a glyco-engineered antibody, and especially a hypofucosylated antibody.

In some embodiments, the said glyco-engineered antibody is directed against a tumor associated antigen.

In some embodiments, the said tumor-associated antigen is selected in a group comprising HER2, HER3, HER4 and AMHRII.

In some embodiments, the said antibody is selected in a group comprising the antibodies termed 3C23K, 9F7F11, H4B121 and HE4B33 disclosed herein, as well as variants thereof.

In some embodiments, the said cancer treatment comprises administering to the said individual a further anti-cancer agent.

In some embodiments, the said cancer treatment comprises administering to the said individual an inhibitory immune checkpoint inhibitor, such as an inhibitor of PD-1, PD-L1, PD-L2, BTLA, CTLA-4, A2AR, B7-H3 (CD276), B7-H4 (VTCN1), IDO, KIR, LAG3, TIM-3, VISTA, CD137, OX40, OX40L and B7S1.

In some embodiments, the said inhibitor consists of an antibody directed against the said inhibitory immune checkpoint, or an antigen-binding fragment thereof.

This invention also pertains to a pharmaceutical composition comprising (i) a glyco-engineered Fc fragment-bearing compound and (ii) an inhibitory immune checkpoint inhibitor.

### DESCRIPTION OF THE FIGURES

FIG. 1: Glyco-engineered 3C23K mAb (GM102) reduces macrophages induced-T cells inhibition.

FIG. 1A: Measure of PBT proliferation co-cultured with MDM2 macrophages targeting COV434-AMHRII tumor cells. MDM2 were challenged with COV434-AMHRII cell line opsonized with either the irrelevant mAb R565 (isotype Ctrl), the anti-AMHRII FcKO or the anti-AMHRII 3C23K for 4 days prior the co-culture for an additional 4 days with anti-CD3/CD28 pre-activated peripheral blood T cells (also termed “PBT”). Data represent the Division Index (i.e. the average number of cell divisions that a cell in the original population has undergone) of pre-activated CD8+ T cells+/−Standard Deviation. (Data are representative of three independent experiments. P-values * <0.05). Abscissa, from the left to the right: (i) n.a. T cells Ctrl, (ii) a. T cells Ctrl, (iii) isotype Ctrl, (iv) FcKO and (v) 3C23K.

FIG. 1B: Measure of PBT proliferation co-cultured with MDM2 macrophages targeting mAb treated polystyrene beads. MDM2 were challenged with polystyrene beads non coated as control or coated with either the anti-AMHRII FcKO or the anti-AMHRII 3C23K for 24 hours prior the co-culture for an additional 4 days with pre-activated cell trace violet loaded PBT. Data represents the Division Index (i.e. the average number of cell divisions that a cell in the original population has undergone) of pre-activated CD8+ T cells+/−Standard Deviation. (Data are representative of three independent experiments. P-values ** <0.01). n.a. refers to non-activated and a. to activated T cells. Abscissa, from the left to the right: (i) n.a. T cells Ctrl, (ii) a. T cells Ctrl, (iii) Untreated beads, (iv) FcKO and (v) 3C23K.

FIG. 2: The glycoengineered 3C23K (GM102) antibody does not affect the proliferation of human T lymphocytes. CellTrace Violet loaded T cells were activated by CD3/CD28 coated beads in presence or absence of 10 μg/ml of the anti-AMHRII 3C23K mAb. 3 days later the dilution of CellTrace Violet was evaluated by flow cytometry and represented as raw data (FIG. 2A) and as the % of T cells that have divided 1, 2, 3 or 4 times (FIG. 2B). In FIG. 2B, ordinate represents the percentage in each peak; from the bottom to the top: (i) 0 division, (ii) 1 division, (iii) 2 divisions, (iv) 3 divisions and (v) 4 divisions.

FIG. 3: Activation of TAM-like macrophages by a Fc-bearing glyco-engineered compound

FIG. 3A illustrates a decrease in the expression of markers associated with macrophages of the M2 phenotype, such as Sepp1 (mRNA—FIG. 3A-1), Stab1 (mRNA—FIG. 3A-2), FOLFR2 (m-RNA—FIG. 3A-3) and CD163 (Protein—FIG. 3A-4).

The boxes on the left illustrate M2 type macrophages grown in wells without antibodies. The central boxes illustrate the type M2 macrophages cultured in wells with FcKO antibody. The boxes on the right illustrate the type M2 macrophages cultured in wells with the low fucosylated R18H2 antibody.

FIG. 3 B-1 (mRNA expression) and FIG. 3B-2 (Protein expression) illustrate an increase in the expression of CD16.

The boxes on the left illustrate M2 type macrophages grown in wells without antibodies. The central boxes illustrate the type M2 macrophages cultured in wells with FcKO antibody. The boxes on the right illustrate the type M2 macrophages cultured in wells with the low fucosylated R18H2 antibody.

FIG. 3 C-1 (mRNA expression), FIG. 3C-2 (mRNA expression) and FIG. 3C-3 (Protein expression) illustrate an increase in the expression of CD64.

The boxes on the left illustrate M2 type macrophages grown in wells without antibodies.

The central boxes illustrate the type M2 macrophages cultured in wells with FcKO antibody. The boxes on the right illustrate the type M2 macrophages cultured in wells with the low fucosylated R18H2 antibody.

FIG. 3D illustrates an increase of pro-inflamatory factors usually expressed by M1 macrophages, such as TNFα (FIG. 3D-1), IL1β (FIG. 3D-2).

The boxes on the left illustrate M2 type macrophages grown in wells without antibodies. The central boxes illustrate the type M2 macrophages cultured in wells with FcKO antibody. The boxes on the right illustrate the type M2 macrophages cultured in wells with the low fucosylated R18H2 antibody.

FIG. 3E illustrates a decrease in mRNA levels of gene and coding immunosuppressing factor TGFβ.

The box on the left illustrates M2 type macrophages grown in wells without antibodies. The central box illustrates the type M2 macrophages cultured in wells with FcKO antibody. The box on the right illustrates the type M2 macrophages cultured in wells with the low fucosylated R18H2 antibody.

FIG. 3F illustrates a decrease in mRNA levels of gene and coding immunosuppressing factor IDO1.

The box on the left illustrates M2 type macrophages grown in wells without antibodies. The central box illustrates the type M2 macrophages cultured in wells with FcKO antibody. The box on the right illustrates the type M2 macrophages cultured in wells with the low fucosylated R18H2 antibody.

FIG. 3G-1 (mRNA expression) and FIG. 3G-2 (Protein expression) illustrate a decrease of immunosuppressing factor IL10.

The boxes on the left illustrate M2 type macrophages grown in wells without antibodies. The central boxes illustrate the type M2 macrophages cultured in wells with FcKO antibody. The boxes on the right illustrate the type M2 macrophages cultured in wells with the low fucosylated R18H2 antibody.

FIG. 3H (mRNA expression) illustrates a decrease of pro-angiogenic factor PDGFα. The box on the left illustrates M2 type macrophages grown in wells without antibodies. The central box illustrates the type M2 macrophages cultured in wells with FcKO antibody. The box on the right illustrates the type M2 macrophages cultured in wells with the low fucosylated R18H2 antibody.

FIG. 3I (mRNA expression) illustrates a decrease of pro-angiogenic factors VEGFβ. The box on the left illustrates M2 type macrophages grown in wells without antibodies. The central box illustrates the type M2 macrophages cultured in wells with FcKO antibody. The box on the right illustrates the type M2 macrophages cultured in wells with the low fucosylated R18H2 antibody.

FIG. 3J (mRNA expression) illustrates a decrease of pro-angiogenic factors HGF. The box on the left illustrates M2 type macrophages grown in wells without antibodies. The central box illustrates the type M2 macrophages cultured in wells with FcKO antibody. The box on the right illustrates the type M2 macrophages cultured in wells with the low fucosylated R18H2 antibody.

FIG. 3K the PDL2 expression at the surface of M2 macrophages.

The white box illustrates the type M2 macrophages cultured in wells with FcKO antibody. The black box illustrates the type M2 macrophages cultured in wells with the low fucosylated R18H2 antibody.

FIG. 4: Glyco-engineered 3C23K (GM102) antibody blocks immunosuppression, which leads to an activation of the immune system.

FIG. 4A: ADCC generated by TAM-like macrophages on SKOV3-AMHRII cells incubated 4 h at 37° C. with anti-AMHRII antibodies. From left to right: 3C23K-FcKO, 3C23K CHO and 3C23K YB20 (means 3C23K YB2/0). Data from 3 donors of macrophages tested in triplicate are presented. FIG. 4B: Cytolysis of SKOV3-AMHRII cancer cells by TAM-like macrophages incubated 4 days with anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4C: Percentage of CD8 memory (CD8+CD25+) macrophages after four days of co-incubation of TAM-like macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4D: Percentage of Th1 (CD4+CD183+) macrophages after four days of co-incubation of TAM-like macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented. FIG. 4E: Percentage of Th2 (CD4+CD183-) macrophages after four days of co-incubation of TAM-like macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4F: Detection of CXCL9 in culture medium after four days of co-incubation of TAM-like macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4G: Detection of CXCL10 in culture medium after four days of co-incubation of TAM-like macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4H: Detection of CCL2 in culture medium after four days of co-incubation of TAM-like macrophages with SKOV3-AMHRII cancer cells and anti-AMHRII antibodies. Due to different base line for each donor, data (in triplicate) of the 3 donors are individualized. For each donor from left to right: 3C23K-FcKO, 3C23K CHO and 3C23K YB20 (means 3C23K YB2/0).

FIG. 4I: Detection of IL1β in culture medium after four days of co-incubation of TAM-like macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4J: Detection of IL6 in culture medium after four days of co-incubation of TAM-like macrophages with SKOV3-AMHRII cancer cells and anti-AMHRII antibodies. From left to right: 3C23K-FcKO, 3C23K CHO and 3C23K YB20. Data from 3 donors of macrophages tested in triplicate are presented.

FIG. 4K: Detection of CCL5 in culture medium after four days of co-incubation of TAM-like macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4L: Detection of IL12 in culture medium after four days of co-incubation of undifferentiated macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4M: Detection of IL6 in culture medium after four days of co-incubation of undifferentiated macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4N: Detection of IL1β in culture medium after four days of co-incubation of undifferentiated macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4O: Detection of IL23 in culture medium after four days of co-incubation of undifferentiated macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4P: Detection of CXCL9 in culture medium after four days of co-incubation of undifferentiated macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 4Q: Detection of CXCL10 in culture medium after four days of co-incubation of undifferentiated macrophages with SKOV3-AMHRII cancer cells and anti-AMHRII antibodies. From left to right: 3C23K-FcKO, 3C23K CHO and 3C23K YB20 (means 3C23K YB2/0). Data from 3 donors of macrophages tested in triplicate are presented.

FIG. 5: Activation of macrophages present in the tumor tissue of cancer patients administered with a glyco-engineered antibody

FIGS. 5A (cd16) and 5B (granzyme): Results of an immuno-fluorescence assay of cell markers performed in a tumor tissue of a cancer patient administered with the 3C23K antibody.

FIG. 5A: Percentage of CD16 Positive Tumor Tissue in the Patient 04-01 and the Patient 01-01 before (baseline) and under treatment. Black box illustrate Patient 04-01 and grey box illustrate Patient 01-01.

FIG. 5B: Percentage of cells in the Patient 01-01 before (baseline) and under treatment. Black box illustrate the percentage of cells CD8+ GZB+/mm2; grey box illustrate percentage of cells CD16+ GZB+/mm2; and white box represent percentage of NKp46+ GZB+/mm2.

FIG. 6: Activation of NK cells, monocytes and ICOS+ T cells in cancer patients administered with a glyco-engineered antibody

FIG. 6A: Quantification by flow cytometry (Mean Flow Intensity) of CD4+ ICOS+ cells in blood samples of patients treated by GM102 (3C23K). Samples were took at cycle 1 before (C1J0) and during (C1J0+4H) injection of GM102 (3C23K), then before cycle 2 and 3 (C2J1 and C3J1 respectively).

FIG. 6B: Quantification by flow cytometry (Mean Flow Intensity) ofCD8+ ICOS+ cells in blood samples of patients treated by GM102 3C23K at Gustave Roussy. Samples were took at cycle 1 before (C1J0) and during (C1J0+4H) injection of GM102 (3C23K), then before cycle 2 and 3 (C2J1 and C3J1 respectively).

FIG. 7: Percentage of classical, intermediate and non-classical monocyte subsets within CD14+ cells in patients after and under treatment.

### DETAILED DESCRIPTION OF THE INVENTION

Using an in vitro model of cancer tissue comprising cancer cells and immune system cells such as T cells and macrophages, the present inventors have found that inhibition of T cell activation may be unexpectedly induced induced by M2 tumor-associated macrophages (TAMs).

Further, the inventors have shown that such a TAMs-induced inhibition of T cell activation may be reduced or blocked by adding glyco-engineered antibodies to this in vitro cancer tissue model. Glyco-engineered antibodies, and especially hypofucolsylated antibodies, are known in the art to bind with a high affinity to Fc receptors and in particular Fc receptors present at the macrophage membrane, especially FcγRIIIa (also termed “CD16a” in the art).

Without wishing to be bound by any particular theory, the inventors believe that the binding of glyco-engineered antibodies to the Fc receptors present at the macrophage membrane induce the release of soluble factors, e.g. the release of cytokines, exerting an inhibition-blocking effect on the T cells present within the tumor tissue environment, or alternatively an activation of the T cells present within the tumor tissue environment. Thus, the inventors believe that glyco-engineered antibodies, by blocking a T cell inhibition or activating T cells, are able to reduce or block the inhibition of the immune response against cancer cells that occurs in certain individuals affected with a cancer.

The inventors have further shown herein that tumor cells themselves are dispensable in the immuno-activation effect that is obtained in the presence of glyco-engineered antibodies.

Still further, the inventors have shown that the said glyco-engineered antibodies, which are shown to bind with a high affinity to the Fc-gamma receptors of TAM-like macrophages, induce TAM-like macrophages of the immunosuppressive M2 phenotype towards the non-immunosuppressive M1 phenotype, with a concomitant reduction of immunosuppressive cytokines such as IL-10. The inventors have also shown that, in the presence of a glyco-engineered antibody as described herein, the said TAM-like macrophages have a higher expression of pro-inflammatory cytokines such as IL-1 beta without a significant change in the expression of pro-tumoral genes such as VEGF alpha, VEGF beta, PDGF beta and Hepatocyte Growth Factor.

It is also shown herein that the administration of a glyco-engineered antibody as described herein induces an increase in the level of CD8+ T cells of a cancer patient. Without wishing to be bound by any particular theory, the inventors believe that the glyco-engineered antibody, because it induces macrophages to release T cells activating cytokines, allows removing the T-cell inhibition occurring in cancer patients undergoing an immunosuppression state, thus leading to an activation of the CD8+ T cells.

Still further, it is shown herein that a glyco-engineered antibody as described herein induces an increase in the CD4+ T cells of the Th1 phenotype and a decrease in the CD4+ T cells of the Th2 phenotype. Such a change in the balance between Th1 and Th2 T-cells is expected to favorize an increase of an immune response against tumor cells.

The inventors have also shown that a glyco-engineered antibody as described herein modulate the expression of cytokines such as IL1 beta, IL6, IL10, IL12 and IL23 It is also shown herein that a glyco-engineered antibody as described herein induces naïve macrophages to lower their production of immunosuppressive cytokines such as IL-10.

The inventors have further shown that the administration of a glyco-engineered antibody as described herein to a cancer patient induces an increase in the number of CD16+(Fc gamma RIII+) cells in the tumor tissue. Thus, the administration of a glyco-engineered antibody as described herein to a cancer patient leads to an increase in anti-tumor activated macrophages within the tumor tissue. It has been further shown that the administration of a glyco-engineered antibody as described herein to a cancer patient increases the level of Granzyme B-producing activated macrophages in the tumor tissue, which inhibition of the immunosuppressive state shall contribute to cytolysis of tumor cells. Still further, the administration of a glyco-engineered antibody as described herein to a cancer patient also increases the number of NK cells in the tumor tissue, which inhibition of immunosuppression shall equally contribute to the killing of tumors cells.

The present inventors have also shown that the administration of a glyco-engineered antibody as described herein to a cancer patient (i) increases the expression of CD16 (Fc gamma RIII) by NK cells, (ii) increases the expression of CD69 on monocytes and (iii) increases expression of ICOS (Inducible T-cell COStimulator) on T cells, which are other parameters that materialize an inhibition of an immunosuppression state undergone by the cancer patients.

Altogether, the inventors' findings show that glyco-engineered antibodies are able to reduce or block the macrophage-induced suppression of T cell anti-tumor activities.

The inventors' results have allowed them to conceive therapeutic tools based on the administration of glyco-engineered antibodies, the said therapeutic tools being aimed at reducing or blocking an immunosuppression state that may occur in individuals affected with a cancer.

Without wishing to be bound by any particular theory, the inventors believe that the immunostimulating effect that is induced by the glyco-engineered antibodies is due to the high affinity of the said glyco-engineered antibodies for the Fc receptors present at the cell membrane, and especially to the Fc receptors present at the macrophage membrane, irrespective of whether the said antibody possess or not a relevant antigen-binding region.

As shown in the examples herein, the reducing or the blocking of the immunosuppression state is obtained even in a model wherein tumor antigen expressing cells are absent, which may mean that the binding of the said glyco-engineered antibodies to a tumor antigen and the reduction of tumor load induced by phagocytosis itself may not be required for inducing their immuno-activation effect, and especially may not be required for reducing or blocking the macrophage-induced T cell inhibition. In other words, the present inventors believe that the blocking of the T cell inhibition by the said glyco-engineered antibodies relates to the behavior of these antibodies as glyco-engineered Fc fragment-bearing compounds.

The present invention relates to a glyco-engineered Fc fragment-bearing compound for its use as an immunosuppression inhibitor in the cancer treatment of an individual.

This invention pertains to a glyco-engineered Fc fragment-bearing compound for its use for preventing or treating an immunosuppression state in an individual affected with a cancer.

This invention concerns the use of a glyco-engineered Fc fragment-bearing compound as an immunosuppression inhibitor for preparing a medicament for treating a cancer.

This invention relates to the use of a glyco-engineered Fc fragment-bearing compound for preparing a medicament for preventing or treating an immunosuppression state in an individual affected with a cancer.

This invention pertains to a method for treating a cancer comprising a step of administering, to an individual in need thereof, a glyco-engineered Fc fragment-bearing compound as an immunosuppression inhibitor.

This invention concerns a method for preventing or treating an immunosuppression state in an individual affected with a cancer, comprising a step of administering, to an individual in need thereof, a glyco-engineered Fc fragment-bearing compound.

This invention relates to a glyco-engineered Fc fragment-bearing compound for its use for reducing or blocking of an immunosuppression state caused by a macrophage-induced T cell inhibition occurring in an individual affected with a cancer.

The present invention pertains to the use of to a glyco-engineered Fc fragment-bearing compound for preparing a medicament for reducing or blocking of an immunosuppression state caused by a macrophage-induced T cell inhibition occurring in an individual affected with a cancer.

This invention also concerns a method for reducing or blocking of an immunosuppression state caused by a macrophage-induced T cell inhibition occurring in an individual affected with a cancer, comprising a step of administering, to an individual in need thereof, a glyco-engineered Fc fragment-bearing compound.

It flows from the preceding embodiments that the cancer individuals that are concerned by the present invention are those which are also affected with an immunosuppression.

In some embodiments, the cancer individuals that are concerned by the present invention are those which are also affected with an immunosuppression that is caused by an anti-cancer treatment.

### Definitions

According to the invention, the expression “comprising”, such as in “comprising the steps of”, is also understood as “consisting of”, such as in “consisting of the steps of”.”

As used herein, the terms “cancer-associated immunosuppression”, “cancer-related immunosuppression”, “immunosuppression state” when related to an individual affected with a cancer, means a physiological state encompassing situations wherein CD8⁺ T cells have their ability to be activated that is reduced or blocked, i.e. have their ability to be activated that is partly or totally inhibited.

Illustratively, an individual affected with a cancer which undergoes an immunosuppression state, according to the present invention, may be determined by an in vitro test method comprising a step of measuring the ability to proliferate of peripheral blood CD8⁺ T cells contained in a sample previously collected from the said individual, which peripheral blood T cells having been subjected to a step of pre-activation before measuring their proliferation capacity.

Thus, in some embodiments, an immunosuppression state may be detected in a tested individual when the ability to proliferate of the CD8+ T cells of the said tested individual is lower than a reference CD8+ T cell proliferation capacity value that is indicative of the absence of an immunosuppression state. In some embodiments, the said reference CD8+ T cell proliferation capacity value may be the mean CD8+ T cell proliferation capacity value that is found in healthy individuals which are not immunosuppressed. In some other embodiments, the said reference value may be a threshold value allowing discriminating between (i) CD8+ T cell proliferation capacity values that are lower (or alternatively higher, depending on the measure units that are used) than the threshold value, which is indicative of an immunosuppression state and (ii) CD8+ T cell proliferation capacity values that are higher (or alternatively lower, depending on the measure units that are used) than the threshold value, which is indicative of the absence of an immunosuppression state.

In some embodiments, the CD8+ T cell proliferation capacity value is the division index value of the CD8+ T cells, as illustrated in the examples herein.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, a glyco-engineered Fc fragment-bearing compound encompasses any compound comprising a Fc fragment of an antibody that possesses an altered glycosylation allowing the binding of the said Fc fragment with a high affinity to Fc receptors, and especially to the Fc receptors present at the macrophage membrane, which includes to the Fc receptors present at the membrane of tumor-associated macrophages.

In some embodiments, the Fc-containing protein comprises one or more polypeptides.

As used herein, “Fc fragment-bearing protein” refers to a protein comprising a Fc fragment fused to at least one other heterologous protein unit or polypeptide.

Glyco-engineered Fc fragment-bearing compounds encompass (i) glyco-engineered Fc fragments themselves, (ii) hybrid compounds comprising a glyco-engineered Fc fragment that is covalently linked to a non-protein moiety and (iii) protein compounds comprising a glyco-engineered Fc fragment that is linked to a protein moiety.

Glyco-engineered Fc fragment bearing protein compounds encompass proteins wherein the said glyco-engineered Fc fragment is covalently linked to an antigen-binding domain of an antibody, such as covalently linked to the variable regions of an antibody.

Glyco-engineered Fc fragment bearing protein compounds encompass proteins wherein the said glyco-engineered Fc fragment is covalently linked, directly or indirectly, to one or more other Fc fragments, such as covalently linked to one or more other glyco-engineered Fc fragments. Illustrative examples of such glyco-engineered Fc fragment-bearing compound encompass compounds known in the art as “Fc multimers”, such as described for example by Thiruppathi et al. (2014, J Autoimmun, Vol. 52: 64-73), by Jain et al. (2012, Arthritis Research and Therapy, Vol. 14: R192), or by Zhou et al. (2017, Blood advances, Vol. 1 (no 6): DOI 10.1182/biooadvances.2016001917).

In some preferred embodiments, glyco-engineered Fc fragment-bearing compounds according to the invention encompass glyco-engineered antibodies.

In some preferred embodiments, glyco-engineered antibodies encompass antibodies directed to a tumor-associated antigen.

As used herein, the term “antibody” refers to such assemblies (e.g., intact antibody molecules, antibody fragments, or variants thereof) which have significant known specific immunoreactive activity to an antigen of interest, and especially an immunoreactive activity to a tumor associated antigen of interest. Antibodies and immunoglobulins comprise light and heavy chains, with or without an interchain covalent linkage between them.

Light chains of immunoglobulins are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarily determining regions” or “CDRs” interspersed among “framework regions” or “FRs”, as defined herein.

The regions of an immunoglobulin heavy or light chain may be defined as “constant” (C) region or “variable” (V) regions, based on the relative lack of sequence variation within the regions of various class members in the case of a “constant region”, or the significant variation within the regions of various class members in the case of a “variable regions”.

By convention the numbering of the variable constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

Amino acid positions in a heavy chain constant region, including amino acid positions in the CH1, hinge, CH2, CH3, and CL domains, may be numbered according to the Kabat index numbering system (see Kabat et al, in “Sequences of Proteins of Immunological Interest”, U.S. Dept. Health and Human Services, 5th edition, 1991). Alternatively, antibody amino acid positions may be numbered according to the EU index numbering system (see Kabat et al, ibid).

As used herein, the term “Fc region” is defined as the portion of a heavy chain constant region beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge domain, a CH2 domain, and a CH3 domain.

The term “Fc fragment” as used herein refers to a molecule comprising the sequence of a non-antigen-binding fragment resulting from digestion of an antibody or produced by other means, whether in monomeric or multimeric form, and can contain the hinge region. The original immunoglobulin source of the Fc fragment can be of human origin and can be any of the immunoglobulins, such as IgG1 or IgG2. Fc fragments are made up of monomeric polypeptides that can be linked into dimeric or multimeric forms by covalent (i.e., disulfide bonds) and non-covalent association. The number of intermolecular disulfide bonds between monomeric subunits of Fc fragments ranges from 1 to 4 depending on class (e.g., IgG, IgA, and IgE) or subclass (e.g., IgG1, IgG2, IgG3, IgA1, and IgGA2). One example of a Fc fragment is a disulfide-bonded dimer resulting from papain digestion of an IgG.

The term “Fc fragment” as used herein is generic to the monomeric, dimeric, and multimeric forms.

As used herein the term “Fc fragment-bearing protein” or “Fc fragment-containing protein” refers to a protein that comprises an Fc domain, or an Fc-receptor binding fragment thereof, comprising an N-glycan. In certain embodiments, the N-glycan is an N-linked biantennary glycans present in the CH2 domain of an immunoglobulin constant (Fc) region (e.g., at EU position 297). “N-glycans” are attached at an amide nitrogen of an asparagine or an arginine residue in a protein via an N-acetylglucosamine residue. These “N-linked glycosylation sites” occur in the peptide primary structure containing, for example, the amino acid sequence asparagine-X-serine/threonine, where X is any amino acid residue except proline and aspartic acid. Such N-Glycans are fully described in, for example, Drickamer K, Taylor M E (2006). Introduction to Glycobiology, 2nd ed., which is incorporated herein by reference in its entirety.

In one embodiment, “N glycan” refers to the Asn-297 N-linked biantennary glycans present in the CH2 domain of an immunoglobulin constant (Fc) region. These oligosaccharides may contain terminal mannose, N-acetyl-glucosamine, Galactose or Sialic acid.

As used herein, the term “glycoengineering” refers to any art-recognized method for altering the glycoform profile of a binding protein composition. Such methods include expressing a binding protein composition in a genetically engineered host cell (e.g., a CHO cell) that has been genetically engineered to express a heterologous glycosyltransferase or glycosidase. In other embodiments, the glycoengineering methods comprise culturing a host cell under conditions that bias for particular glycoform profiles.

As used herein, a “glyco-engineered Fc fragment” encompasses (i) a hyper-galactosylated Fc fragment, (ii) a hypo mannosylated Fc fragment, which encompasses a amannosylated Fc fragment, and (iii) a hypo fucosylated Fc fragment, which encompasses a afucosylated Fc fragment. As used herein, a glyco-engineered fragment encompasses a Fc fragment having an altered glycosylation which is selected in a group comprising one or more of the following altered glycosylation (i) hyper-galactosylation, (ii) hypo-mannosylation and (iii) hypo-fucosylation. Consequently, a glyco-engineered Fc fragment as used according to the invention encompass the illustrative examples of a hyper-galactosylated, a hypo-mannosylated and a hypo-fucosylated Fc fragment.

The one skilled in the art may refer to well-known techniques for obtaining hyper-galactosylated Fc fragments, hypo mannosylated Fc fragments and hypo fucosylated Fc fragments that are known to bind to Fc receptors with a higher affinity than non-modified Fc fragments.

As used herein the term “hypergalactosylated population” refers to a population of Fc domain-containing binding proteins in which the galactose content of the N glycan is increased as compared to a reference population of Fc domain-containing binding proteins having the same amino acid sequence. A hypergalactosylated population can be expressed as having an increased number of G1 and G2 glyco forms as compared to the reference population of Fc domain-containing binding proteins.

As used herein, the term “hypomannosylated population” refers to a population of Fc domain-containing binding proteins in which the mannose content of the N glycan is decreased as compared to a reference population of Fc domain-containing binding proteins having the same amino acid sequence. A hypomannosylated population can be expressed as having a decreased number of oligomannose glycoforms (e.g., M3-M9 glycoforms) as compared to the reference population of Fc domain-containing binding proteins. In some embodiments, the mannose content is determined by measuring the content of one or more of oligomannose glycoforms selected from the group consisting of Man3, Man4, Man5, Man 6, Man 7, Man 8 and Man 9. In other embodiments, the oligomannose content is determined by measuring at least Man 5, Man 6, and Man 7. In certain embodiments, the oligomannose content is determined by measuring all M3-M9 glycoforms. As used herein the terms “GO glyco form,” “G1 glyco form,” and “G2 glyco form” refer to N-Glycan glycoforms that have zero, one or two terminal galactose residues respectively. These terms include GO, G1, and G2 glycoforms that are fucosylated or comprise a bisecting N-acetylglucosamine residue. In certain embodiments, the G1 and G2 glycoforms further comprise sialic acid residues linked to one or both of the terminal galactose residues to form G1S1, G2S1 and G2S2 glycoforms. As used herein the terms “GiS 1 glycoform,” “G2S1 glycoform,” and “G2S2 glycoform” refer to N-Glycan glycoforms that have a sialic acid residue linked to the sole terminal galactose residue in a G1 glycoform, one of the terminal galactose residue in a G2 glycoform, or both of the terminal galactose residue in a G2 glycoform, respectively. These terms include G1S1, G2S1 and G2S2 glycoforms that are fucosylated or comprise a bisecting N-acetylglucosamine residue. In certain embodiments, the sialic residues of G1S1, G2S1 and G2S2 glycoforms are linked by alpha-2,6-sialic acid linkages to the terminal galactose residue of each glycoform in order to enhance the anti-inflammatory activity of the binding molecule (see e.g., Anthony et al., PNAS 105: 19571-19578, 2008).

The definition of “hypofucosylated” or “afucosylated” below, as applied to specific embodiments of a glyco-engineered Fc-bearing compound consisting of an antibody, is relevant for the generality of the glyco-engineered Fc-bearing compounds of interest.

A “hypofucosylated” antibody preparation refers to an antibody preparation in which less than 50% of the N-linked oligosaccharide chains contain α1,6-fucose attached to the CH2 domain. Typically, less than about 40%, less than about 30%, less than about 20%, less than about 10%, or less than 5% or less than 1% of the N-linked oligosaccharide chains contain α1,6-fucose attached to the CH2 domain in a “hypofucosylated” antibody preparation. As used herein, an antibody preparation in which less than 50% of the N-linked oligosaccharide chains contain α1,6-fucose attached to the CH2 domain encompasses a preparation wherein less than 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or less than 1% of the N-linked oligosaccharide chains contain α1,6-fucose attached to the CH2 domain.

Thus, the terms “afucosylated” and “non-fucosylated” are used interchangeably herein to refer to an antibody that lacks α1,6-fucose in the carbohydrate attached to the CH2 domain of the IgG heavy chain. Umana et al, Nat. Biotechnol 17:176-180, 1999, which describes bisected GlcNac resulting in 10 times ADCC. Umana notes that such bisected molecules result in less fucosylation. Davies, et al., Biotechnol. Bioeng. 74:288-294, 2001 describe CHO cells with inserted enzyme β1-4-N-acetylglucosaminyltransferase III (GnTIII) (which causes the bisected GlcNac structure) resulting in increased ADCC of anti-CD20 antibodies. Illustratively, the U.S. Pat. No. 6,602,684 describes cells engineered to produce bisecting GlcNac glycoproteins.

Further examples of methods to reduce fucosylation of an antibody preparation are provided in Shields et al, J Biol Chem 277:26733-26740, 2002, which describes CHO cells (Lec13) deficient in fucosylation to produce IgG1 and further describes that binding of the fucose-deficient IgG1 to human FcgammaRIIIA was improved up to 50-fold and increased ADCC. In addition, Shinkawa et al., J Biol Chem 278:3466-3473, 2003; compare IgG produced in YB2/0 and CHO cells. The YB2/0 cells have decreased fucosylation and increased bisecting GlcNac content. Niwa et al., Clinc. Cancer Res. 1-:6248-6255, 2004 compare anti-CD20 antibodies with antibodies made in YB2/0 cells (low fucosylation) and observed enhanced ADCC in the latter. Examples of techniques to produce afucosylated antibodies are provided, for example, in Kanda et al, Glycobiology 17:104-118, 2006. U.S. Pat. No. 6,946,292 (Kanda) describes fucosyltransferase knock-out cells to produce afucosylated antibodies. The U.S. Pat. No. 7,214,775 and the PCT application no WO 00/61739 describe antibody preparations in which 100% of the antibodies are afucosylated.

Still further techniques to modify glycosyation are also known, such as those described in the United States patent applications no US 2007/248600; US 2007/178551 (GlycoFi technology methods employing engineered lower eukaryotic cells (yeast) to produce “human” glycosylation structures); US 2008/060092 (Biolex technology methods employing engineered plants to produce “human” glycosylation structures) and US 2006/253928 (which also described engineering of plants to produce “human” antibodies).

Additional techniques for reducing fucose include ProBioGen technology (von Horsten et al., Glycobiology, (advance access publication Jul. 23, 2010); Potelligent™ technology (Biowa, Inc. Princeton, N.J.); and GlycoMAb™ glycosylation engineering technology (GLYCART biotechnology AG, Zurich, Switzerland).

The N-linked oligosaccharide content of an antibody can be analyzed by methods known in the art. The following is an example of such a method: Antibodies are subjected to digestion with the enzyme N-glycosidase F (Roche; TaKaRa). Released carbohydrates are analyzed by matrix assisted laser desorption/ionization time-of-flight mass spectrometry (MALDI-TOF MS) with positive ion mode (Papac et al., Glycobiol. 8: 445-454, 1998).

Monosaccharide composition is then characterized by modified high-performance anion exchange chromatography (HPAEC) (Shinkawa et al., J. Biol. Chem. 278: 3466-3473, 2003).

In certain embodiments, the glyco-engineered Fc fragment-bearing compounds of the invention are produced in a cultured mammalian host cell line (e.g., a CHO cell line). In certain embodiments, the host cell line has been glycoengineered to produce the hypergalactosylated and/or hypomannosylated binding proteins of the invention. In certain exemplary embodiments, the binding proteins of the invention are obtained from a glycoengineered CHO cell. In one exemplary embodiment, the glycoengineered CHO cell contains a heterologous galactosyltransferase gene (e.g., mouse galactosyltransferase Beta 1,4). In another exemplary embodiment, the glycoengineered CHO cell contains a knockdown of one of the alleles of the Beta galactosidase gene.

According to the present disclosure, the term “3C23K” means Anti-AMHRII humanised monoclonal antibody 3C23K. AMHRII may be also named MSRII.

According to the present disclosure, the term “GM102” means an anti-AMHRII humanised antibody having the light and heavy chains having the same amino acid sequences than the 3C23K antibody but has been glyco-engineered, and more particularly is hypofucosylated. “GM102” may also be termed “R18H2” herein.

According to the present disclosure “YB2/0 cells” (EMABling®) or “YB20” means cell lines for the manufacturing of recombinant monoclonal hypofucolsylated antibodies.

According to the present disclosure, 3C23K-CHO consists of 3C23K antibody with normal glycosylation, which encompasses the 3C23K antibody which has been produced by CHO cell lines.

According to the present disclosure, 3C23K-FcKO consists of 3C23K antibody devoid of the Fc fragment.

Glyco-Engineered Fc Fragment-Bearing Compounds

The terms “glyco-engineered Fc fragment-bearing compound”, “glyco-engineered Fc fragment-bearing molecule”, “glyco-engineered Fc fragment-containing compound” and “glyco-engineered Fc fragment-containing molecule” may be used interchangeably herein for meaning a compound that comprises a Fc fragment of an antibody that has an altered glycosylation providing to the said Fc fragment a higher affinity for a Fc receptor as compared to the same Fc fragment having an unaltered glycosylation.

In some preferred embodiments, a glyco-engineered Fc fragment-bearing compound has a higher affinity for the FcγRIIIa (also termed “CD16a”) than the same Fc fragment that has not undergone glyco-engineering.

This is illustrated in the examples by the hypofucolsylated Fc fragment-bearing compound termed “3C23K” that has a high affinity for the human FcγRIIIa (CD16a), with a Kd constant value, as measured with the well-known Biacore® method, of less than 50 nM.

In some preferred embodiments, the glyco-engineered Fc fragment-bearing compound consists of a glyco-engineered Fc fragment itself, thus a compound that does not comprise an antigen binding region.

In some preferred embodiments, the glyco-engineered Fc fragment-bearing compound consists of a glyco-engineered Fc fragment-bearing protein wherein the said glyco-engineered Fc fragment is covalently linked to another protein moiety, that is either (i) a protein comprising an antigen binding region or (ii) a protein which does not comprise an antigen binding region.

In some of these preferred embodiments, the said glyco-engineered Fc fragment-bearing compound comprises only one glyco-engineered Fc fragment.

Thus, this invention encompasses the use of glyco-engineered Fc fragment-bearing compounds comprising (i) a polypeptide monomer unit comprising a glyco-engineered Fc fragment and (ii) another polypeptide which is covalently linked to the said polypeptide monomer unit. The said another polypeptide may be an antigen-binding region of an antibody, such as the VH and VL chains of an antibody. The said another polypeptide may be a ligand-binding protein moiety, such as a receptor protein, like for example a VEGF receptor or a VEGF-binding domain of a VEGF receptor, or like for example a TNF alpha receptor or a TNF-binding domain of a TNF alpha receptor.

In some of these preferred embodiments, the said other protein moiety may comprise another Fc fragment, and especially another glyco-engineered Fc fragment. In some embodiments, the two glyco-engineered Fc fragments have identical amino acid sequences. In some other embodiments, the two glyco-engineered Fc fragments have distinct amino acid sequences. In some embodiments, the two Fc fragments have identical amino acid sequences but have distinct altered glycosylation patterns. In some other embodiments, the two Fc fragments have identical amino acid sequences and have an identical altered glycosylation pattern.

Thus, glyco-engineered Fc fragment-bearing compounds encompass protein compounds comprising more than one Fc fragment, provided that at least one of the Fc fragments comprised therein is glyco-engineered, such as at least one of the Fc fragments comprised therein is hypo-mannosylated, hyper galactosylated or hypo-fucosylated.

As already mentioned elsewhere in the present specification, Fc fragment-bearing compounds comprising more than one Fc fragment, such as comprising two, three, four, five or six Fc fragments, are well known in the art and may be termed “Fc multimers”. Such Fc multimers constructs are disclosed notably by Thiruppathi et al. (2014, J Autoimmun, Vol. 52: 64-73), by Jain et al. (2012, Arthritis Research and Therapy, Vol. 14 R192), or by Zhou et al. (2017, Blood advances, Vol. 1 (no 6): DOI 10.1182/biooadvances.2016001917).

Thus, glyco-engineered Fc-bearing compounds that may be used according to the invention encompass a multimeric fusion protein comprising two or more polypeptide monomer units (i) wherein each polypeptide monomer unit comprises a Fc fragment and (ii) wherein at least one polypeptide monomer unit comprises a glyco-engineered Fc fragment, such as comprises a hypo-mannosylated Fc fragment, a hyper-galactosylated Fc fragment or a hypo-fucosylated Fc fragment.

Such Fc multimers are also disclosed in the United States patent application no US 2017/088063.

In some embodiments of Fc multimer compounds, the said compounds also comprised therein an antigen-binding domain, such as for example those disclosed by Zhang et al. (2016, J Immunol, Vol. 196:1165-1176).

In some other preferred embodiments, the glyco-engineered Fc fragment-bearing compound consists of a glyco-engineered antibody, as it is illustrated in the examples herein.

In some preferred embodiments, the glyco-engineered Fc fragment-bearing compound consists of a hypofucosylated Fc fragment-bearing compound, such as a hypofucosylated antibody, as it is illustrated in the examples herein.

In some other embodiments, the glyco-engineered Fc fragment-bearing compound, and more precisely the hypo-fucosylated Fc fragment-bearing compound, consists of a afucosylated Fc fragment-bearing compound, such as a afucosylated antibody.

In other embodiments, the glyco-engineered Fc fragment-bearing compound consists of a hypergalactosylated Fc fragment-bearing compound, such as a hypargalactosylated antibody.

In still other embodiments, the glyco-engineered Fc fragment-bearing compound consists of a hypomannosylated Fc fragment-bearing compound, such as a hypomannosylated antibody.

As already described elsewhere in the present specification, reducing or blocking of an immunosuppression, such as a marcrophage-induced immunosuppression, occurring during a cancer disease by a glyco-engineered Fc fragment-bearing compound, such as a glyco-engineered antibody, does not require the presence of tumor cells, and thus does not require the binding of the said antibody to target tumor cells.

This explains why the inventors believe that reducing or blocking immunosuppression, and especially inhibition of the activation of T cells, shall be obtained by glyco-engineered Fc fragment-bearing compounds that do not comprise an antigen-binding region, such as do not comprise a tumor-associated antigen-binding region.

However, it is also illustrated in the examples herein that reducing or blocking immunosuppression is reached when using glyco-engineered antibodies as glyco-engineered Fc fragment-bearing compounds.

Moreover, the present inventors believe that the beneficial effects of a glyco-engineered Fc fragment-bearing compound defined herein may be further increased when using a glyco-engineered Fc fragment-bearing compound consisting of a glyco-engineered antibody directed against a relevant tumor-associated antigen, which means a glyco-engineered antibody directed against a tumor-associated antigen that is expressed by the tumor cells present in the tumor tissue or in the body fluids of the cancer individual to be treated.

Thus, in some preferred embodiments, the glyco-engineered Fc fragment-bearing compound consists of a glyco-engineered antibody directed against a tumor-associated antigen expressed by the tumor cells of the cancer individual to be treated.

In some preferred embodiments, the said glyco-engineered antibody consists of a hypofucosylated antibody, as it is illustrated in the examples herein.

Without wishing to be bound by any particular theory, the inventors believe that using a glyco-engineered antibody directed against a tumor-associated antigen expressed by the tumor cells of the cancer individual to be treated (i) allows reducing or blocking the immunosuppression, such as an inhibition of T cells activation, particularly an inhibition of CD8+ T cells activation, such as a macrophage-induced immunosuppression, and (ii) allows destruction of the tumor cells expressing the tumor-associated antigen against which the said glyco-engineered antibody is directed, such as by an ADCC or ADC activity.

The term “tumour associated antigen” as used herein refers to an antigen that is or can be presented on a surface that is located on or within tumour cells. These antigens can be presented on the cell surface with an extracellular part, which is often combined with a transmembrane and cytoplasmic part of the molecule. These antigens can in some embodiments be presented only by tumour cells and not by normal, i.e. non-tumour cells. Tumour antigens can be exclusively expressed on tumour cells or may represent a tumour specific mutation compared to non-tumour cells. In such an embodiment a respective antigen may be referred to as a tumour-specific antigen or tumor-associated antigen (also termed “TAA”). Some antigens are presented by both tumour cells and non-tumour cells, which may also be referred to as tumour-associated antigens. These tumour-associated antigens can be overexpressed on tumour cells when compared to non-tumour cells or are accessible for antibody binding in tumour cells due to the less compact structure of the tumour tissue compared to non-tumour tissue. In some embodiments the tumour associated surface antigen is located on the vasculature of a tumour.

A list of tumour-associated antigens is disclosed notably by Liu et al. (2016, European Journal of Cancer Care, doi: 10/1111/ecc.12446), to which the one skilled in the art may refer.

A list of tumour antigens recognized by T cells is disclosed by Renkvist et al. (2001, Cancer immunology and immunotherapy, Vol. 50 (no 1) 3-15), to which the one skilled in the art may also refer.

Illustrative examples of a tumor associated surface antigen are CD10, CD19, CD20, CD22, CD33, Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2neu, Her3, IGFR, CD133, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-a (CD140a), PDGFR-β (CD140b) Endoglin, CLEC14, Teml-8, and Tie2. Further examples may include A33, CAM PATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRvlll, EpCAM, Ep-CAM, Folate-binding protein, G250, Fms-like tyrosine kinase 3 (FLT-3, CD135), c-Kit (CD1 17), CSF1 R (CD1 15), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

Preferred Tumor-associated antigens (TAAs) may be selected in a group comprising CD45, IL-3Ra (also termed CD123), CD33, CD20, CD22, CD19, EpCAM (also termed “Epithelial Cell Adhesion Molecule”), HER2, TROP-2 (also termed “Trophoblast cell surface antigen 2”), GNMB (also termed “Glyco-protein non-metastatic B”), MMP9, EGFR, PD-L1 (CD274), CTLA4, GM3, Mesothelin, Folate receptor 1, Fibronectin extradomain B, Endoglin, CD22, IL-1 alpha, HER3, cMet, Phosphatidylserine, MUC5AC, NeuGc gangliosides, CD2, CD38, EGFR, HGF/SF, PD1, GD2, ST4 and Folate receptor alpha.

Most preferred tumor-associated antigens according to the present invention are those selected in a group comprising HER2, HER3, HER4 and AMHRII.

Preferred embodiments of glyco-engineered antibodies that may be used according to the present invention are selected in a group comprising the glyco-engineered antibodies termed 3C23K, 9F7F11, H4B121 and HE4B33 herein Illustrative embodiments of a glyco-engineered Fc fragment-bearing compound Illustrative embodiments of Fc-fragment-bearing compounds encompass compounds comprising a glyco-engineered Fc Fragment comprising two amino acid chains of SEQ ID NO. 70 described herein.

The amino acid chain of SEQ ID NO. 70 consists of the heavy chain constant region of a human IgG1 antibody, comprising the CH1 domain, the Hinge region, the CH2 domain and the CH3 domain.

As it is disclosed in the examples, a glyco-engineered Fc fragment-bearing compound, and especially a hypofucolsylated Fc fragment-bearing compound, may be obtained by a method comprising a step of expression of the nucleic acid sequence encoding the said Fc fragment in YB2/0 cells. Such a method may be the well-known method termed EMABling®, which is described in the examples.

In some embodiments, a glyco-engineered Fc fragment-bearing compound, and especially a hypofucolsylated Fc fragment-bearing compound, may be obtained by a method comprising a step of expression of the nucleic acid sequence of SEQ ID NO. 69 in YB2/0 cells.

In some embodiments, the said glyco-engineered Fc fragment-bearing compound consists of a glyco-engineered antibody, and especially a hypofucolsylated antibody; comprising the said glyco-engineered Fc fragment comprising two amino acid chains of SEQ ID NO. 70.

Embodiments of glyco-engineered antibodies comprising a glyco-engineered Fc fragments are described hereunder.

Antibodies as glyco-engineered Fc fragment-bearing compounds

Thus, embodiments of a glyco-engineered Fc fragment-bearing compound consist of antibodies, and especially glyco-engineered antibodies directed against tumor-associated antigens.

In some embodiments, glyco-engineered Fc fragment-bearing compounds encompass glyco-engineered multi-specific antibodies and especially glyco-engineered bispecific antibodies. Illustratively, those glyco-engineered antibodies encompass antibodies comprising a glyco-engineered Fc fragment as described herein and (i) a first antigen binding region that binds to a tumor antigen and (ii) a second antigen binding region that binds to a T cell antigen such as the CD3 or an inhibitory immune checkpoint protein, e.g. with the view of simultaneously (i) target tumor antigen expressing cells and (ii) activating T cells.

Illustrative examples of such glyco-engineered antibodies encompass those which are directed against tumor-associated antigens such as AMHRII, HER2, HER3 and HER4.

Such antibodies may be described in relation to their antigen-binding regions, and especially their heavy chain variable region (VH) and light chain variable region (VL).

Illustrative Embodiments of Anti-AMHRII Antibodies

The PCT application no PCT/FR2011/050745 (International Publication no WO/2011/141653) and U.S. Pat. No. 9,012,607, each of which is hereby incorporated by reference in its entirety, disclose novel humanized antibodies that are derived from the murine 12G4 antibody. These humanized antibodies may be used as AMHRII-binding agents for the purpose of the present invention. In particular embodiments disclosed in the PCT application no WO/2011/141653, the antibodies are those identified as the 3C23 and 3C23K. The nucleic acid sequences and polypeptide sequences of these antibodies are provided as SEQ ID NOs: 1-16 herein. In some aspects of the invention, the anti-AMHRII antibodies of interest may be referred to as “comprising a light chain comprising SEQ ID NO: and a heavy chain comprising SEQ ID NO:”. Thus, in various embodiments, particularly preferred antibodies, comprise:
a) a light chain comprising SEQ ID NO: 2 and a heavy chain comprising SEQ ID NO: 4 (3C23 VL and VH sequences without leaders);
b) a light chain comprising SEQ ID NO: 6 and a heavy chain comprising SEQ ID NO: 8 (3C23K VL and VH sequences without leaders);
c) a light chain comprising SEQ ID NO: 10 and a heavy chain comprising SEQ ID NO: 12 (3C23 light and heavy chains without leaders);
d) a light chain comprising SEQ ID NO: 14 and a heavy chain comprising SEQ ID NO: 16 (3C23K light and heavy chains without leaders).

Other antibodies (e.g., humanized or chimeric antibodies) can be based upon the heavy and light chain sequences described herein.

Illustrative embodiments of anti-AMHRII antibodies comprising/containing CDRs comprising (or consisting of) the following sequences:

CDRL-1:
(SEQ ID NO: 71)
RASX1X2VX3X4X5A,
where X1 and X2 are, independently, S or P, X3 is
R or W or G, X4 is T or D, and X5 is I or T;
CDRL-2 is
(SEQ ID NO: 72)
PTSSLX6S
where X6 is K or E; and
CDRL-3 is
(SEQ ID NO: 73)
LQWSSYPWT;
CDRH-1 is
(SEQ ID NO: 74)
KASGYX7FTX8X9HIH
where X7 is S or T, X8 is S or G and X9 is Y or N;
CDRH-2 is
(SEQ ID NO: 75)
WIYPX10DDSTKYSQKFQG
where X10 is G or E
and
CDRH-3 is
(SEQ ID NO: 76)
GDRFAY.

Antibodies (e.g., chimeric or humanized) within the scope of this application include those disclosed in the following table: 3C23K antibody is defined by:
SEQ ID NO: 17 for VH amino acid sequence
SEQ ID NO: 34 for VL amino acid sequence

Table 1 hereunder lists anti-AMHRII humanized antibodies that may be used according to the invention.

TABLE 1
anti-AMHRII antibodies

| | Mutations | | | |
|---|---|---|---|---|
| | | SEQ ID in | | SEQ ID in |
| | VH | sequence | | sequence |
| Antibody | mutations | listing | VL mutations | listing |
| 3C23K | | 17 | | 34 |
| 3C23 | | 17 | L-K55E | 35 |
| 3C23KR | H-R3Q | 18 | | 34 |
| 6878 | H-R3Q | 18 | L-T481, L-P50S | 36 |
| 5842 | H-R3Q, | 19 | L-T481, L-K55E | 37 |
| | H-T73A | | | |
| K4D-24 | H-Q1R | 20 | | 34 |
| 6C59 | H-Q1R | 20 | L-527P, L-528P | 38 |
| K4D-20 | H-Y32N | 21 | | 34 |
| K4A-12 | H-A16T | 22 | | 34 |
| K5D-05 | H-531G | 23 | | 34 |
| K5D-14 | H-T285 | 24 | | 34 |
| K4D-123 | H-R445 | 25 | | 34 |
| K4D-127 | H-169T | 26 | | 34 |
| 6C07 | H-169T | 26 | L-M4L, L-T20A | 39 |
| 5C14 | H-169F | 27 | | 34 |
| 5C26 | H-V67M | 28 | L-527P | 40 |
| 5C27 | H-L45P | 29 | | 34 |
| 5C60 | H-E10K, | 30 | | 34 |
| | H-K12R | | | |
| 6C13 | H-G53E | 31 | | 34 |
| 6C18 | H-T93A | 32 | | 34 |
| 6C54 | H-584P | 33 | L-M4L, L-59P, | 41 |
| | | | L-R31W | |
| K4D-25 | | 34 | L-M4L | 42 |
| K4A-03 | | 35 | L-133T | 43 |
| K4A-08 | | 36 | L-M4L, L-K39E | 44 |
| K5D-26 | | 37 | L-T22P | 45 |
| 5C08 | | 38 | L-Y32D | 46 |
| 5C10 | | 17 | L-S27P | 40 |
| 5C18 | | 17 | L-Q37H | 47 |
| 5C42 | | 17 | L-G975 | 48 |
| 5C44 | | 17 | L-512P | 49 |
| 5C52 | | 17 | L-19A | 50 |
| 5C56 | | 17 | L-T72A | 51 |
| 6C03 | | 17 | L-R31W | 52 |
| 6C05 | | 17 | L-M4L, L-M39K | 53 |
| 6C16 | | 17 | L-12N | 54 |
| 6C17 | | 17 | L-G63C, L-W91C | 55 |
| 6C28 | | 17 | L-R31G | 56 |
| 725C02 | | 17 | L-175F | 57 |
| 725C17 | | 17 | L-12T | 58 |
| 725C21 | | 17 | L-12T, L-K42R | 59 |
| 725C33 | | 17 | L-Y49H | 60 |
| 725C42 | | 17 | L-M4L, L-T205, | 61 |
| | | | L-K39E | |
| 725C44 | | 17 | L-527P | 40 |
| 725C57 | | 17 | L-T69P | 62 |

Illustrative Embodiments of Anti-HER3 Antibodies

Illustrative embodiments of glyco-engineered anti-HER3 antibodies are those that are termed 9F7F11 and H4B121 herein.

9F7F11 antibody comprises (i) a heavy chain variable region of SEQ ID NO. 63 and (ii) a light chain variable region of SEQ ID NO. 64.

H4B121 antibody comprises (i) a heavy chain variable region of SEQ ID NO. 65 and (ii) a light chain variable region of SEQ ID NO. 66.

Illustrative Embodiments of Anti-HER4 Antibodies

An illustrative embodiment of an anti-HER4 antibody is the antibody which is termed HE4B33 herein.

HE4B33 antibody comprises (i) a heavy chain variable region of SEQ ID NO. 67 and (ii) a light chain variable region of SEQ ID NO. 68 For the sake of clarity, the said above-described antibodies all comprise a glyco-engineered Fc fragment as described herein, and especially comprise a hypofucolsylated Fc fragment as described herein.

In some preferred embodiments, these antibodies comprise a glyco-engineered Fc fragment having two glyco-engineered amino acid chains of SEQ ID NO. 70, and especially a hypofucolsylated Fc fragment having two hypofucolsylated amino acid chains of SEQ ID NO. 70.

Combination of a Glyco-Engineered Fc Fragment-Bearing Compound with One or More Other Active Agents

A glyco-engineered Fc fragment-bearing compound as defined herein, because it allows reducing or blocking an immunosuppression state in cancer patients, is useful to potentiate the anti-cancer activity of known anti-cancer treatments, which include surgical treatments, radiotherapy treatments and chemotherapy treatments.

Further, a glyco-engineered Fc fragment-bearing compound as defined herein, because it allows reducing or blocking an immunosuppression state in cancer patients, is thought to possibly act as an active agent that shall increase the beneficial effects of other compounds aimed at blocking immunosuppression or aimed at inducing an immune-stimulation or an immuno-activation in immunosuppressed cancer patients. Moreover glyco-engineered Fc fragment-bearing compounds could also contribute to act against resistance of cancer cells to those immunosuppression inhibitors (check point inhibitors) or immuno stimulating agents.

Thus, in further aspects, a glyco-engineered Fc fragment-bearing compound as defined herein may be used in combination with another anti-cancer treatment, and in particular in combination with one or more distinct compounds consisting of anti-cancer agents.

In a further aspect, the present invention relates to a glyco-engineered Fc fragment-bearing compound for its use as an immunosuppression inhibitor in the cancer treatment of an individual, in combination with one or more distinct anti-cancer agents.

This invention further relates to the use of a glyco-engineered Fc fragment-bearing compound in combination with one or more distinct anti-cancer agents for preparing a medicament for treating a cancer.

This invention also pertains to a method for treating a cancer comprising a step of administering, to an individual in need thereof, a glyco-engineered Fc fragment-bearing compound in combination with one or more distinct anti-cancer agents.

Anti-cancer agents encompass compounds that possess an anti-cancer activity such as antiproliferative active agents, wherein a high number of these are well-known from the one skilled in the art. Anti-cancer agents also encompass inhibitors of inhibitory immune checkpoint proteins, as it is detailed elsewhere in the present specification.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer.

In some embodiments, the said cancer agents do not consist of antibody-derived compounds, such as antibodies themselves or antigen-binding fragments or antigen-binding formats thereof.

Examples of anti-cancer agents which do not consist of antibodies include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin I1, or rlL.sub.2), interferon alfa-2a; interferon alfa-2b; interferon alfa-nl; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™) erlotinib (Tarceva™) cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like. Details of administration routes, doses, and treatment regimens of anti-cancer agents are known in the art, for example as described in “Cancer Clinical Pharmacology” (2005) ed. By Jan H. M. Schellens, Howard L. McLeod and David R. Newell, Oxford University Press.

In some other embodiments, the said further anti-cancer agents consist of anti-cancer antibodies which are distinct from the one or more Fc-bearing compound which is used for inhibiting a cancer-related immunosuppression. Anti-cancer antibodies encompass monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), radioimmunotherapy (e.g., anti-CD20 monoclonal antibody conjugated to ¹¹¹In, ⁹⁰Y, or ¹³¹I, etc.). In some embodiments, these anti-cancer antibodies may be themselves glyco-engineered, such as hypofucosylated.

Anti-cancer agents also encompass agents that are known to activate or reactivate the anti-cancer activity of the immune system. Agents that activate or reactivate the anti-cancer activity of the immune system encompass those which are preferably those which inhibit inhibitory immune checkpoints. These agents may be termed herein “inhibitory immune checkpoint inhibitors” or “immune checkpoint inhibitors”. As it is known in the art, an immune checkpoint inhibitor consists of an agent that inhibits the activity of inhibitory immune checkpoint proteins.

The term “immune checkpoint protein” is known in the art. Within the known meaning of this term it will be clear to the skilled person that on the level of “immune checkpoint proteins” the immune system provides inhibitory signals to its components in order to balance immune reactions. Known immune checkpoint proteins comprise CTLA-4, PD1 and its ligands PD-L1 and PD-L2 and in addition LAG-3, BTLA, B7H3, B7H4, TIM3, KIR. The pathways involving LAG3, BTLA, B7H3, B7H4, TIM3, and KIR are recognized in the art to constitute immune checkpoint pathways similar to the CTLA-4 and PD-1 dependent pathways (see e.g. Pardoll, 2012. Nature Rev Cancer 12:252-264; Mellman et al, 2011. Nature 480:480-489).

Within the present invention an immune checkpoint protein inhibitor is any compound inhibiting the function of an immune checkpoint protein. Inhibition includes reduction of function and full blockade. In particular, the immune checkpoint protein is a human immune checkpoint protein. Thus, the immune checkpoint protein inhibitor preferably is an inhibitor of a human immune checkpoint protein. Immune checkpoint proteins are described in the art (see for instance Pardoll, 2012. Nature Rev. cancer 12: 252-264). The designation immune checkpoint protein includes the experimental demonstration of stimulation of an antigen-receptor-triggered T lymphocyte response by inhibition of the immune checkpoint protein in vitro or in vivo, e.g. mice deficient in expression of the immune checkpoint protein demonstrate enhanced antigen-specific T lymphocyte responses or signs of autoimmunity (such as disclosed in Waterhouse et al, 1995. Science 270:985-988; Nishimura et al, 1999. Immunity 11:141-151). It may also include demonstration of inhibition of antigen-receptor triggered CD4⁺ or CD8⁺ T cell responses due to deliberate stimulation of the immune checkpoint protein in vitro or in vivo (e.g. Zhu et al, 2005. Nature Immunol. 6:1245-1252). Preferred immune checkpoint protein inhibitors are antibodies that specifically recognize immune checkpoint proteins. A number of CTLA-4, PD1, PDL-1, PD-L2, LAG-3, BTLA, B7H3, B7H4, TIM3 and KIR inhibitors are known and in analogy of these known immune checkpoint protein inhibitors, alternative immune checkpoint inhibitors may be developed in the (near) future. For example ipilimumab is a fully human CTLA-4 blocking antibody presently marketed under the name Yervoy (Bristol-Myers Squibb). A second CTLA-4 inhibitor is tremelimumab (referenced in Ribas et al, 2013, J. Clin. Oncol. 31:616-22). Examples of PD-1 inhibitors include without limitation humanized antibodies blocking human PD-1 such as lambrolizumab (e.g. disclosed as hPD109A and its humanized derivatives h409All, h409A16 and h409A17 in WO2008/156712; Hamid et al, N. Engl. J. Med. 369: 134-144 2013), or pidilizumab (disclosed in Rosenblatt et al, 2011. J Immunother. 34:409-18), as well as fully human antibodies such as nivolumab (previously known as MDX-1106 or BMS-936558, Topalian et al, 2012. N. Eng. J. Med. 366:2443-2454, disclosed in U.S. Pat. No. 8,008,449). Other PD-1 inhibitors may include presentations of soluble PD-1 ligand including without limitation PD-L2 Fc fusion protein also known as B7-DC-Ig or AMP-244 (disclosed in Mkrtichyan M, et al. J Immunol. 189:2338-47 2012) and other PD-1 inhibitors presently under investigation and/or development for use in therapy. In addition, immune checkpoint inhibitors may include without limitation humanized or fully human antibodies blocking PD-L1 such as MEDI-4736 (disclosed in WO2011066389), MPDL328 OA (disclosed in U.S. Pat. No. 8,217,149) and MIH1 (Affymetrix obtainable via eBioscience (16.5983.82)) and other PD-L1 inhibitors presently under investigation. According to this invention an immune checkpoint inhibitor is preferably selected from a CTLA-4, PD-1 or PD-L1 inhibitor, such as selected from the known CTLA-4, PD-1 or PD-L1 inhibitors mentioned above (ipilimumab, tremelimumab, labrolizumab, nivolumab, pidilizumab, AMP-244, MEDI-4736, MPDL328 OA, MIH1). Known inhibitors of these immune checkpoint proteins may be used as such or analogues may be used, in particular chimeric, humanized or human forms of antibodies.

As the skilled person will know, alternative and/or equivalent names may be in use for certain antibodies mentioned above. Such alternative and/or equivalent names are interchangeable in the context of the present invention. For example, it is known that lambrolizumab is also known under the alternative and equivalent names MK-3475 and pembrolizumab.

The selection of an immune checkpoint inhibitor from PD1 and PD-L1 inhibitors, such as a known PD-1 or PD-L1 inhibitor mentioned above, is more preferred and most preferably a selection is made from a PD-1 inhibitor, such as a known PD1 inhibitor mentioned above. In preferred embodiments, the PD1 inhibitor is nivolumab or pembrolizumab or another antagonist antibody against human PD1.

The invention also includes the selection of other immune checkpoint inhibitors that are known in the art to stimulate immune responses. This includes inhibitors that directly or indirectly stimulate or enhance antigen-specific T-lymphocytes. These other immune checkpoint inhibitors include, without limitation, agents targeting immune checkpoint proteins and pathways involving PD-L2, LAG3, BTLA, B7H4 and TIM3. For example, human PD-L2 inhibitors known in the art include MIH18 (disclosed in Pfistershammer et al., 2006. Eur J Immunol. 36:1104-13). Another example, LAG3 inhibitors known in the art include soluble LAG3 (IMP321, or LAG3-Ig disclosed in WO2009044273, and in Brignon et al., 2009. Clin. Cancer Res. 15:6225-6231) as well as mouse or humanized antibodies blocking human LAG3 (for instance IMP701 disclosed in and derived from WO2008132601), or fully human antibodies blocking human LAG3 (such as disclosed in EP 2320940). Another example is provided by the use of blocking agents towards BTLA, including without limitation antibodies blocking human BTLA interaction with its ligand (such as 4C7 disclosed in WO2011014438). Yet another example is provided by the use of agents neutralizing B7H4 including without limitation antibodies to human B7H4 (disclosed in WO 2013025779 A1, and in WO 2013067492) or soluble recombinant forms of B7H4 (such as disclosed in US20120177645 or Anti-human B7H4 clone H74: eBiocience #14-5948). Yet another example is provided by agents neutralizing B7-H3, including without limitation antibodies neutralizing human B7-H3 (e.g. MGA271 disclosed as BRCA84D and derivatives in US 20120294796). Yet another example is provided by agents targeting TIM3, including without limitation antibodies targeting human TIM3 (e.g. as disclosed in WO 2013006490 or the anti-human TIM3, blocking antibody F38-2E2 disclosed by Jones et al., J Exp Med. 2008 Nov. 24; 205 (12): 2763-79). Known inhibitors of immune checkpoint proteins may be used in their known form or analogues may be used, in particular, chimeric forms of antibodies, most preferably humanized forms.

The invention also includes the selection of more than one immune checkpoint inhibitor selected from CTLA-4, PD-1 or PDL1 inhibitors for combination with a glyco-engineered Fc fragment-bearing compound within the various aspects of the invention. For example concurrent therapy of ipilimumab (anti-CTLA4) with Nivolumab (anti-PD1) has demonstrated clinical activity that appears to be distinct from that obtained in monotherapy (Wolchok et al., 2013, N. Eng. J. Med., 369:122-33). Also included are combinations of agents that have been shown to improve the efficacy of checkpoint inhibitors, such as Lirilumab (also known as anti-KIR, BMS-986015 or IPH2102, as disclosed in U.S. Pat. No. 8,119,775 and Benson et al., Blood 120:4324-4333 (2012)) in combination with ipilimumab (Rizvi et al., ASCO 2013, and clinicaltrials.gov NCT01750580) or in combination with nivolumab (Sanborn et al., ASCO 2013, and clinicaltrials.gov NCT01714739), agents targeting LAG3 combined with anti-PD-1 (Woo et al., 2012 Cancer Res. 72:917-27) or anti-PD-L1 (Butler N S et al., Nat Immunol. 2011 13:188-95), agents targeting ICOS in combination with anti-CTLA-4 (Fu et al, Cancer Res. 2011 71:5445-54, or agents targeting 4-1BB in combination with anti-CTLA-4 (Curran et al., PLoS One. 2011 6 (4) el 9499).

According to the present invention, preferred targeted inhibitory immune checkpoint proteins encompass those selected in a group comprising PD-1, PD-L1, PD-L2, BTLA, CTLA-4, A2AR, B7-H3 (CD276), B7-H4 (VTCN1), IDO, KIR, LAG3, TIM-3 and VISTA.

Preferred inhibitors of an inhibitory immune checkpoint protein of interest disclosed herein consist of antibodies directed against the said inhibitory immune checkpoint protein of interest and which inhibit the activity of the said inhibitory immune checkpoint protein of interest.

Thus, in some preferred embodiments, inhibitors of inhibitory immune checkpoint proteins that may be used according to the present invention encompass those selected in a group comprising antibodies directed to one of PD-1, PD-L1, PD-L2, BTLA, CTLA-4, A2AR, B7-H3 (CD276), B7-H4 (VTCN1), IDO, KIR, LAG3, TIM-3 and VISTA.

Cancers within the present invention include, but are not limited to, leukemia, acute lymphocytic leukemia, acute myelocytic leukemia, myeloblasts promyelocyte, myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endothelio sarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

Pharmaceutical Compositions and Therapeutic Methods

As already described elsewhere in the present specification, a glyco-engineered Fc fragment-bearing compound as defined herein may be advantageously used in the course of a combined treatment with one or more further anti-cancer therapies, and especially in the course of a combined treatment with one or more further anti-cancer agents, which includes in the course of a combined treatment with one or more inhibitory immune checkpoint protein inhibitors.

According to these embodiments, the said glyco-engineered Fc fragment-bearing compound and the said further anti-cancer agent(s) are “co-administered”.

The term “co-administration” as used herein refers to the administration of at least two different substances sufficiently close in time to modulate an immune response. Preferably, co-administration refers to simultaneous administration of at least two different substances.

Thus, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another.

In some embodiments, the selected glyco-engineered Fc fragment-bearing compound and the one or more further anti-cancer agent(s) are administered simultaneously to the cancer individual to be treated, and the two active agents may be comprised in the same pharmaceutical composition or alternatively may be comprised in separate pharmaceutical compositions. These two separate pharmaceutical compositions may be mixed together before use and then administered to the cancer individual to be treated. In other embodiments, these two separate pharmaceutical compositions may be administered to the cancer individual to be treated at short time interval, e.g. within 2-5 minutes time interval.

This invention further relates to a pharmaceutical composition comprising (i) a glyco-engineered Fc fragment-bearing compound and (ii) one or more distinct anti-cancer agents.

This invention encompasses a pharmaceutical composition comprising (i) a glyco-engineered Fc fragment-bearing compound and (ii) one or more inhibitory immune checkpoint protein inhibitors.

In some preferred embodiments, the said glyco-engineered Fc fragment-bearing compound is a glyco-engineered antibody directed against a tumor antigen.

In some embodiments, the tumor antigen is selected in the group consisting of HER2, HER3, HER4 and AMHRII.

In some embodiments, the said glyco-engineered antibody is selected in the group consisting of the glyco-engineered antibodies termed 3C23K or a variant thereof, 9F7F111, H4B121 and HE4B33, which are disclosed in detail elsewhere in the present specification.

In some embodiments, the said inhibitory immune checkpoint protein inhibitor is selected in the group consisting of inhibitors of PD-1, PD-L1, PD-L2, BTLA, CTLA-4, A2AR, B7-H3 (CD276), B7-H4 (VTCN1), IDO, KIR, LAG3, TIM-3 and VISTA.

In some embodiments, the said inhibitor consists of an antibody directed against the said inhibitory immune checkpoint protein, or an antigen-binding fragment thereof.

Methods of preparing and administering glyco-engineered Fc-bearing compounds and more generally polypeptides of the current disclosure to a subject are well known to or are readily determined by those skilled in the art. The route of administration of the polypeptides of the current disclosure may be oral, parenteral, by inhalation or topical. The term parenteral as used herein includes intravenous, intraarterial, intraperitoneal, intramuscular, subcutaneous, rectal or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the glyco-engineered Fc-bearing compounds can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glyco, polyethylene glyco, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M or 0.05M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage, and should also be preserved against the contaminating action of microorganisms, such as bacteria and fungi. The carrier can be a solvent or dispersion medium containing, for example, water, ethanol, polyol (e.g., glycerol, propylene glyco, and liquid polyethylene glyco, and the like), and suitable mixtures thereof. The proper fluidity can be maintained, for example, by the use of a coating such as lecithin, by the maintenance of the required particle size in the case of dispersion and by the use of surfactants.

In any case, sterile injectable solutions can be prepared by incorporating an active compound (e.g., a glyco-engineered Fc fragment-bearing compound by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation typically include vacuum drying and freeze-drying, which yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in the patent applications U.S. Ser. Nos. 09/259,337 and 09/259,338 each of which is incorporated herein by reference.

Effective doses of the compositions of the present disclosure, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

Glyco-engineered Fc fragment-bearing compounds of the current disclosure can be administered on multiple occasions. Intervals between single dosages can be weekly, monthly or yearly. Intervals can also be irregular as indicated by measuring blood levels of the said glyco-engineered Fc fragment-bearing compound in the patient. In some methods, dosage is adjusted to achieve a plasma glyco-engineered Fc fragment-bearing compound concentration, and especially of a glyco-engineered antibody concentration, of 1-1000 μg/ml and in some methods 25-300 μg/ml. Alternatively, glyco-engineered Fc fragment-bearing compounds can be administered as a sustained release formulation, in which case less frequent administration is required. For glyco-engineered antibodies, dosage and frequency vary depending on the half-life of the antibody in the patient. In general, humanized antibodies show the longest half-life, followed by chimeric antibodies and nonhuman antibodies.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the glyco-engineered Fc fragment-bearing compound shall be held to mean an amount sufficient to achieve effective to achieve a benefit, e.g., to reduce or block an immunosuppression state occurring in a cancer patient. Of course, the pharmaceutical compositions of the present disclosure may be administered in single or multiple doses to provide for a pharmaceutically effective amount of the glyco-engineered Fc fragment-bearing compound.

### Examples

### Example 1: Synthesis of a Glyco-Engineered Fc Fragment-Bearing Compound

A. Materials and Methods

Cloning of Chimeric 12G4, Humanized 12G4 and 3C23K

Chimeric 12G4 (ch12G4) was constructed and expressed as described previously (27). Briefly, the V_{L} and V_{H} DNA sequences were subcloned sequentially into the polycistronic CHK622-08 vector that contains the promoter, Kozak sequence and the sequences of the human Kappa/IgG1 constant regions.

The DNA sequences coding for humanized 12G4 (h12G4) V_{L} and V_{H} were synthesized using Genscript and then cloned in CHK622-08 by digestion and ligation as described above, resulting in the HK622-18 vector. The DNA sequences coding for affinity-matured 3C23K V_{L} and V_{H} were obtained by directed mutagenesis of the phage clone 3C23 to introduce the V_{L} E68K mutation. Signal peptides were added by PCR assembly using the humanized variable regions of h12G4 as template and then cloned in HK622-18, as described above. The resulting vector that expresses the humanized and affinity-matured 3C23K antibody was called HK622-18 MAO 3C23K.

Production and Purification of ch12G4, h12G4 and 3C23K

The different molecules were stably expressed, as previously described (Siberil et al., 2006, Clin Immunol Orlando Fla., Vol. 118: 170-179). CHO-S, HEK293 or YB2/0 cells were stably transfected with the appropriate linearized expression vectors. Ch12G4, h12G4 and 3C23K antibodies were produced in YB2/0 cell using EMS (Invitrogen), 5% Ultra-low IgG fetal calf serum (FCS) (PAA) and 0.5 g/l G418 for 5 to 7 days. 3C23K-CHO-S was produced in CHO-S cells using ProCHO4 (Lonza), 4 mM glutamine and 1 g/l G418 for 7 days.

MAbs were purified from culture supernatants by affinity chromatography using protein A sepharose (GE-Healthcare). The levels of aggregates and endotoxins were determined by gel filtration on Superdex HR/200 (GE-Healthcare) and by LAL testing, respectively. Antibody quality and purity were monitored by SDS-PAGE and Coomassie staining. In addition, the glycosylation patterns and core fucose percentage of each purified antibody were determined by high performance capillary electrophoresis laser induced fluorescence (HPCE-Lif) (51).

SPR Analysis

SPR analyses were performed on a Bia3000 or T200 apparatus at 25° C. in HBS-EP (GE Healthcare). For affinity measurements, MISRII was covalently immobilized (1000 RU) on CM5 sensor chip using EDC/NHS activation according to the manufacturer's instructions (GE Healthcare). Different concentrations (0.5-128 nM) of 12G4 or 3C23K were injected on immobilized receptor during 180 seconds. After 400 seconds of dissociation in running buffer, the sensor chip was regenerated using Gly-HCl pH 1.7. The K_{D} values, taking into account affinity and avidity, were calculated using a Langmuir 1:1 fitting model (BiaEvaluation3.2, GE Healthcare). Antibody-FcγR measurements were performed by single-cycle titration at 100 μl/min on FcγR (Sigma) captured on anti-His (R&D Systems) covalently immobilized at 4000-5000 RU level. The gamma receptor was injected at 20 nM during 60 seconds and five increasing antibody concentrations were injected (injection time=60 seconds). After a dissociation step of 600 seconds in running buffer, sensor surfaces were regenerated using 5 μl of Glycine-HCl pH 1.7. Kinetic parameters were evaluated from the sensorgrams using a heterogeneous Ligand or steady-state fitting models on the T200 evaluation software 3.0 (GE healthcare). All sensorgrams were corrected by subtracting the low signal from the control reference surface (without any immobilized protein) and buffer blank injections before fitting evaluation.

Antibodies

The murine anti-MISRII MAb 12G4 was described by Salhi et al. and Kersual et al. (17,22). Anti-idiotype factor VIII chimeric IgG1 R565 EMABling® MAb and anti-CEA MAb 35A7 (17) were used as irrelevant antibodies.

Results

Chimerization, Humanization and Affinity Maturation

The 3C23K humanized antibody was initially derived from the variable regions of the murine 12G4 MAb (Sahli et al., 2004, Biochem J, Vol. 379: 785-793). The humanization procedure included CDR grafting (MAb h12G4) and affinity maturation by random mutagenesis and phage display, leading to the final molecule 3C23K.

In the first step, candidate human templates for CDR grafting were identified by separately entering the sequences of the V_{L} and V_{H} domains in the IMGT/DomainGapAlign search program (28) and by restricting the search to human sequences in IMGT/GENE-DB (29). The closest human V_{H} gene, IGHV1-3*01, showed 67.34% of identity with the murine counterpart. This identity rose up to 92.85% after grafting the murine 12G4 CDR-IMGT into the human FR-IMGT. The closest human V_{L} gene, IGK1-9*01, showed 62.76% of identity with the murine counterpart. However, IGKV1-5*01 was preferred because the IMGT/GeneFrequency tool (28) indicated that IGK1-9*01 is not very frequently expressed. IGKV1-5*01 has an identity of 58.51% with the V_{L} of 12G4 that was increased to 88.29% after grafting.

To better define the binding characteristics, clone 3C23K was reformatted as an IgG1 antibody, produced in YB2/0 cells and analyzed by surface plasmon resonance (SPR). The 3C23K antibody exhibited a higher binding affinity (K_{D}=5.5×10⁻¹¹ M) than mouse 12G4 (K_{D}=7.9×10⁻¹⁰ M). This later value was very close to the value published in the initial description of the MAb 12G4 (K_{D}=8.6×10⁻¹⁰ M) (22). The gain of binding affinity was also confirmed by flow cytometry using COV434-MISRII cells.

3C23K Production in YB2/0, CHO or HEK293 Cells, Glycosylation Analysis and Effect on Binding to Fcγ Receptors

Oligosaccharide analysis of 3C23K expressed in YB2/0 (EMABling® version; 3C23K) (27), CHO-S(3C23K-CHO) or HEK293 (3C23K-HEK293) cells (used as comparators for functional assays) revealed two clearly different glycosylation patterns. The percentages of fucosylated, galactosylated and bisecting GlcNAc isoforms were 33.0%, 57.2% and 1.8% for 3C23K and 94.6%, 54.4% and 2.0%, for 3C23K-CHO, respectively. The effect of these glycosylation differences on the binding to FcγRs was analyzed by SPR. Binding affinity for hFcγRIIIa and hFcγRIIIb was clearly increased following fucose reduction (1-12 nM and 86.0 nM for 3C23K compared with 31-164 nM and 378 nM for 3C23K-HEK293, respectively), but not for the other FcγRs (hFcγRI, hFcγRIIa, hFcγRIIb) (See also Table 2 in Example 2 hereunder).

Then, 3C23K was expressed in YB2/0 cells using the EMABling® technology to increase the antibody interaction with the low/medium affinity Fc receptor CD16 that is mainly expressed on NK cells and macrophages (Siberil et al., 2006, Clin Immunol Orlando Fla., Vol. 118: 170-179). This property is related to the lower expression of the Fut8 gene in rat myeloma YB2/0 cells compared with other commonly used cell lines, such as CHO cells ((Siberil et al., 2006, Clin Immunol Orlando Fla., Vol. 118: 170-179).

As expected, 3C23K-YB2/0 displayed higher binding affinity for CD16 than high-fucose content 3C23K.

### Example 2: Glycan Analysis of the 3C23K (GM102) Antibody

Background:

GM102 is a humanised monoclonal antibody produced in YB2/0 cells (rat hybridoma YB2/3HL) using clone 18H2.

Carbohydrate moieties are located at ASN₂₉₅ of the heavy chain.

a) Glycan analysis results for GM102:—

TABLE 2

| | | | | Bis- | | |
|---|---|---|---|---|---|---|
| | Potency | Fucose | Galactose | GlcNac | Sialyation | |
| Batch | Specification | | | | | |
| No. | 70-130% | <30% | For info | For info | For info | Process |
| PB01 | 88 | 23 | 24 | 18 | 7 | Process A |
| PB02 | 84 | 13 | 21 | 12 | 9 | |
| PB03 | 83 | 24 | 26 | 16 | 9 | |
| PB04 | 94 | 21 | 27 | 15 | 9 | |
| PB05 | 94 | 21 | 25 | 17 | 5 | |
| CB02 | 79 | 24 | 26 | 17 | 6 | |
| PB06 | 69 | 41 | 17 | 13 | 0 | Process B |
| PB07 | 89 | 17 | 11 | 10 | 5 | |
| PB08 | 88 | 15 | 12 | 9 | 0 | |
| CB03 | 101 | 11 | 17 | 16 | 4 | |

b) PBO1 Reference Standard Characterisation

Analysis of these carbohydrate residues by UPLC-HILIC-FD after N-deglycosylation by PNGase F and tagging of the released carbohydrate residues, revealed the presence of 6 major carbohydrate moieties:

| | |
|---|---|
| 1. non-fucosylated (G0) | 51.1% |
| 2. fucosylated (G0F) | 12.8% |
| 3. mono-galactosylated non-fucosylated (G1) | 10.2% |
| 4. mono-galactosylated fucosylated (G1F) | 4.3% |
| 5. aglycosylated non-fucosylated (G0B) | 9.9% |
| 6. fucosylated with a bisecting GlcNAc (G0FB) | 3.4% |
| Total fucosylated residues was 23% | |

TABLE 3
Comparability of main N-glycosylation forms in PB#01 ref. and LC#02

| Structure (%) | Gam-OF20150730-09-PR18 | GAM-LP01-REF |
|---|---|---|
| G0-Gn | 0.8 | 0.8 |
| G0F-Gn | 0.4 | 0.4 |
| G0 | 49.7 | 51.1 |
| G0B | 9.3 | 9.8 |
| G0F | 13.2 | 12.9 |
| G0FB | 3.1 | 3.4 |
| G1(1,6) | 7.7 | 6.8 |
| G1(1,3) | 3.6 | 3.3 |
| G1(1,6)B | 1.6 | 1.5 |
| G1(1,3)B | 0.0 | 0 |
| G1(1,6)F | 3.7 | 3.1 |
| G1(1,3)F | 1.3 | 1.2 |
| G1(1,6)FB | 1.2 | 1.1 |
| G1(1,3)FB | 0.0 | 0 |
| G2 | 0.5 | 0.4 |
| G2B | 0.8 | 0.8 |
| G2F | 0.4 | 0.4 |
| G2FB | 0.5 | 0.6 |
| G1(1,3)NeuAc1 | 0.9 | 0.9 |
| G1(1,6)FNeuAc1 | 0.0 | 0 |
| G1(1,3)FNeuAc1 | 0.0 | 0 |
| G1(1,3)FBNeuAc1 | 0.0 | 0 |
| G2(1,3)NeuAc1 | 0.6 | 0.3 |
| G2(1,3)BNeuAc1 | 0.0 | 0 |
| G2F(1,3)NeuAc1 | 0.0 | 0 |
| G2FB(1,3)NeuAc1 | 0.0 | 0 |
| G2NeuAc2 | 0.0 | 0 |
| G2BNeuAc2 | 0.0 | 0 |
| G2FNeuAc2 | 0.0 | 0 |
| G2FBNeuAc2 | 0.0 | 0 |
| Structures identifiées (%) | 99.3 | 98.8 |
| Taux de fucosylation (%) | 24 | 23 |
| Indice de galactosylation (%) | 26 | 23 |
| Taux de Bis-GlcNAc (%) | 17 | 17 |
| Taux de sialylation (%) | 6 | 5 |
| Structures sialylées (%) | 2 | 1 |

### Example 3: High Affinity for Fc Receptors of Glyco-Engineered Fc Fragment-Bearing Compounds

A. Materials end Methods

Surface Plasmon Resonnance (SPR) analysis:

Anti-histidine antibodies (R&D Systems) were immobilized on a T200 apparatus at 25° C. in HBS-EP at 10 μl/min flow rate on a CM5 sensor chip using EDC/NHS activation, according to the manufacturer's instructions (GE Healthcare). They were covalently immobilized at the 6900RU level on the flowcell Fc2 and a control reference surface (flowcell Fc1) was prepared using the same chemical treatment but without anti-His antibodies.

All kinetic measurements in Fc1 and Fc2 were performed by single-cycle titration on a T200 apparatus at 25° C. in HBS-EP at 100 μl/min. Each human gamma receptor (R&D Systems) was captured on immobilized anti-His antibodies at 20 nM during 60 s. Five increasing concentrations of antibody were injected (injection time=120 s). After a dissociation step of 600 s in running buffer, sensor surfaces were regenerated using 5 μl of glycine-HCl pH1.7. All the sensorgrams were corrected by subtracting the low signal from the control reference surface and buffer blank injections. Kinetic parameters were evaluated from the sensorgrams using a heterogeneous ligand or two states models from the T200 evaluation software.

B. Results

The results of the measure of the affinity constants (Kd) of the hypo-fucosylated anti-AMHRII 3C23K antibody for the human Fc receptors are depicted in Table 4 below.

TABLE 4

| Receptor | human |
|---|---|
| Fcγ RI/CD64 | 0.2-3.3** |
| Fcγ RII/CD32a | 120* |
| Fcγ RIIbc/CD32bc | 459* |
| Fcγ RIIIa/CD16a | 1.3-46** |
| Fcγ RIIIb/hCD16b | 49* |
| Fcγ RIV/mCD16-2 | — |

Affinity constants are expressed as KD in nM.
*KD was calculated by using the heterogeneous ligand fitting model.
**KD values were determined with the two state reaction model when the fitting did not adhere to the heterogeneous ligand model.

### Example 4: A Reduced Fucose Antibody Blocks Tumor-Associated Macrophage-Induced Immunosuppression in Cancer

A. Materials and Methods

In Vitro Immunological Assay:

T cell proliferation assay was performed as follows. Briefly, CMFDA stained COV434-AMHRII were treated 1 h at 4° C. with 10 μg/ml of either the irrelevant mAb R565 or the anti-AMHRII FcKO, or the anti-AMHRII 3C23K mAb and incubated with unstained MDM2 for 4 days prior addition of CellTrace Violet (Molecular Probes®, Life Technologies™) stained T cells pre-activated by CD3/CD28 Dynabeads at MDM2:T cell ratio of 1:8. After 4 days of additional incubation period, cells were harvested and stained with anti-CD8 PerCP, CD11b PE-Cy7, and CD4 AF647 (BD Pharmingen®) before flow cytometry analysis. Dead cells were excluded by Fixable Viability Dye eFluor® 506 (eBioscience®) staining prior antibodies stainings. T cell proliferation was analyzed on CD8+(CD11b−) T gated cells by the measure of CellTrace Violet dilution corresponding to cells divisions. The Division Index equivalent to the average number of cell divisions that a cell in the original population has undergone was calculated with FlowJo (TreeStar, version 7.6.5). The Division Index equivalent to the average number of cell divisions that a cell in the original population has undergone was calculated.

B. Results

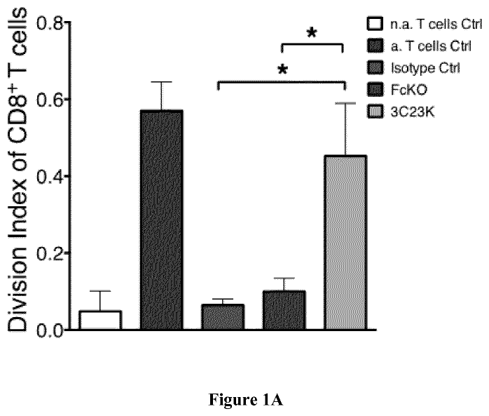

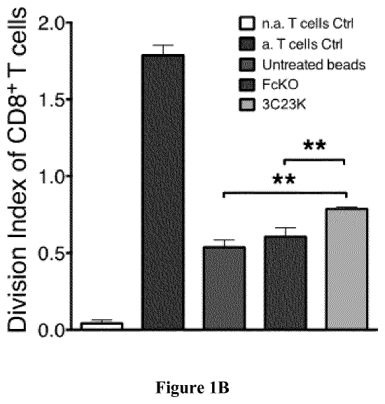

It is clearly established that macrophages within tumors suppress T cell anti-tumor activities. We made the hypothesis that the engagement of macrophages with 3C23K anti-AMHRII antibody alters their T cell suppressive function. To test this hypothesis, COV434-AMHRII target cells were treated with either the irrelevant mAb R565, the anti-AMHRII FcKO or the anti-AMHRII 3C23K mAb and co-cultured with MDM for 4 days prior addition of CD3/CD28 pre-activated PBT. CD8+ T cell proliferation was analyzed by the flow cytometry. As expected, in the presence of control mAbs (irrelevant isotype control R565 and FcKO anti-AMHRII mAbs) or in absence of treatment, MDM strongly impaired T cell proliferation. Of note, MDM mediated T cell immunosuppression was significantly reduced when co-cultured tumor cells were treated with 3C23K anti-AMHRII mAb as shown by the high increase of the division index of CD8 T cells (FIG. 1A).

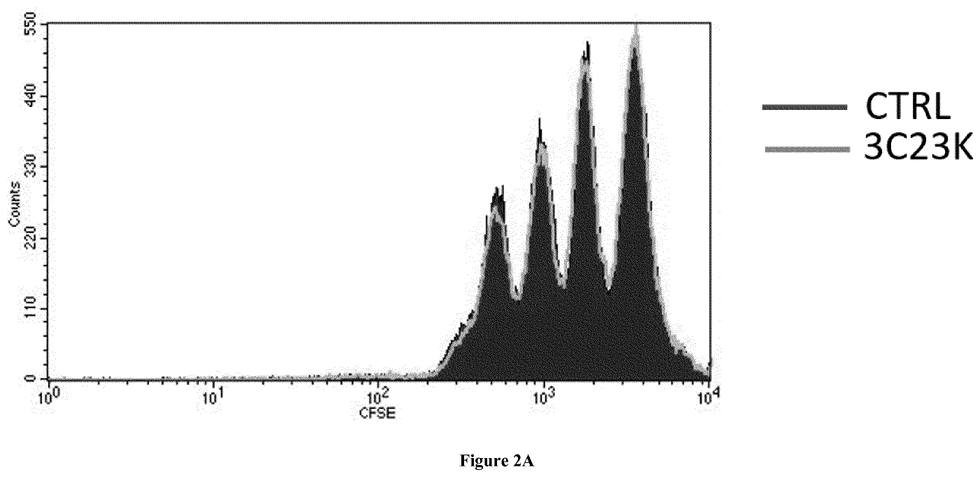

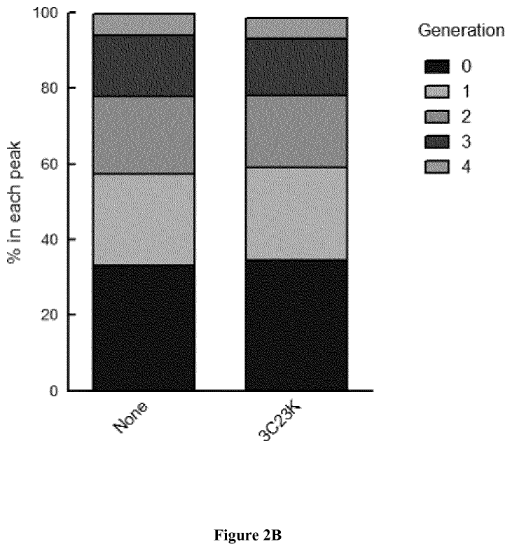

The decrease in tumor cell number can partially explain this “immunostimulating” effect, as tumor cells are known to directly exert T cell suppressive functions. To test whether 3C23K anti-AMHRII mAb could also acts on MDM, rendering them less immunosuppressive we designed an experiment without tumor cells. Inert Sphero® polystyrene beads were used as a substitute for tumor target cells. Those beads were treated with mAb in the same setting of tumors cells i.e. MDM were first co-cultured with mAbs treated beads prior co-culture with activated PBT. In these conditions, CD8+ T cell proliferation was partially restored when MDM were co-cultured with 3C23K coated Sphero® polystyrene beads (FIG. 1B). As a control, we checked that the T cell proliferation observed in the absence of MDM was not affected by 3C23K (FIG. 2). These experiments strongly suggest that 3C23K directly alters the T cell suppressive capacity of MDM.

Together, these results demonstrate that the humanized glyco-engineered monoclonal anti-AMHRII antibody, 3C23K, efficiently targets tumor cells by the antigen binding site and directs pro-tumor macrophages against tumor cells by the recognition of the Fc domain. Thus, mAb activated macrophages trigger ADCC and ADCP against tumor cells and reduced their immunosuppressive behavior towards T cells.

Discussion of the Results

ADCC/ADCP might not be the only mechanism induced by macrophages upon mAb treatment. Tumor-associated macrophages have been described to suppress T cell activation ‘Biswas et al., 2010, Nat. Immunol., Vol. 11 (no 10): 889-896) and our data showing contacts between lymphocytes and macrophages support the idea of direct crosstalk between both cell types. By using in vitro assays, we found that the engagement of FcR by 3C23K decreases the immunosuppressive phenotype of macrophages. In such conditions, pre-activated T cells regain their proliferative capacity that was blocked in the absence of 3C23K. The notion that therapeutic mAbs can engage innate but also adaptive immune cells is consistent with previous studies. In mouse tumor models, it was demonstrated that treatment with anti-tumor antigens Ab induced a cellular immune response, involving T cells, which was required for long-term survival (Montalvao et al., 2013, J Clin Invest, Vol. 123: 5098-5103; Gill et al., 2014, J clin Invest, Vol. 124: 812-823). However, induction of adaptive immune responses in cancer patients that have been treated with anti-tumor mAbs has not yet been extensively investigated.

The mechanism by which 3C23K changes the phenotype of macrophages relieving T cell suppression is not known at present. However, different hypothesis can be envisioned. The interaction of antibodies with Fc receptors expressed by macrophages has been shown to trigger several signaling cascades that regulate the function of these cells (Biswas et al., 2010, Nat. Immunol., Vol. 11 (no 10): 889-896). Our preliminary data show that macrophages activated via FcR with 3C23K produce several pro-inflammatory cytokines including IL-1 beta and IL-6 that have been described to exert beneficial effect on T cells (Grugan et al., 2012, J Immunol., Vol. 189: 5457-5466). Indirect effects are also possible. In particular, the death of tumor cells can lead to the release of several danger-associated molecular pattern molecules (DAMPs) such as calreticulin which in turn activates innate and adaptive immune cells (Yatim et al., 2017, Nat Rev Immunol, Vol. 17 (no 4): 262-275). The role of dendritic cells in mediating this immunogenic cell death has been well described. Evidence also suggests that calreticulin released during cell death activates macrophages which produce IL-6 and TNF-α susceptible to exert beneficial effects on T cells (Duo et al., 2014, Int J Mol Sci, Vol. 15 (no 2): 2916-2928).

### Example 5: Activation of TAM-Like Macrophages by a Fc-Bearing Glyco-Engineered Compound

A. Materials and Methods

Preparation of Human Monocyte-Derived Macrophages

Peripheral blood mononuclear cells (PBMCs) were obtained from healthy blood donors PBMCs were isolated with a classical using positive magnetic selection of CD14+ cells. Monocytes were cultured at 37° C. 5% CO₂ in RPMI supplemented with 10% fetal calf serum then differentiated in M2 type macrophages by addition of 50 ng/mL M-CSF for 4 days. Phenotype of converted M2 type macrophages is CD14high CD163high IL10high IL12low.

In vitro activation of Macrophages by Antibodies

A 10 μg/mL solution of antibody, a low fucosylated anti-AMHRII named R18H2 or its FcKO counterpart without any binding to Fcγ receptors, was adsorbed onto 24-wells plates by an incubation at 4° C. for 24 hours. This experimental condition mimicked a situation where antibodies recognized its antigen. Non-coated antibodies were discarded by washing with PBS solution. M2 type macrophages (10⁶ cells/mL of culture medium) were then incubated at 37° C. for 1 to 3 days in wells coated with antibodies (or not for negative control).

Analysis of Macrophages after Activation by Antibodies

Macrophages incubated with antibodies are stimulated by 100 ng/mL LPS for 6 or 24 hours before analysis by, respectively, qRT-PCR or flow cytometry. Transcription of PDGFα, VEGFP, HGF, TGFβ, IDO1, IL10, Sepp1, Stab1, FOLR2, CD64a, CD64b and CD16a genes were quantified and normalized by using RPS18, B2M and EF1a genes as references.

Variation of expression was confirmed at protein level by flow cytometry for membranous proteins expressed by macrophages and, for soluble factors such IL10, IL1β or TNFα by a classical ELISA assay with samples from culture media.

B. Results

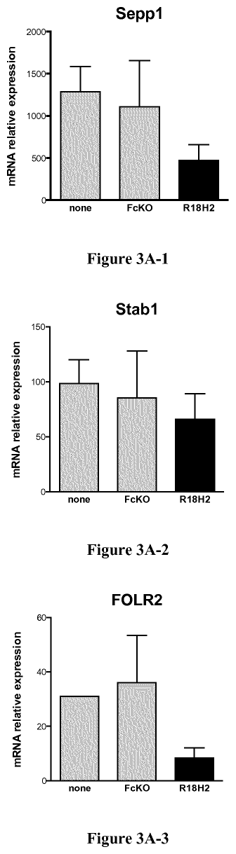

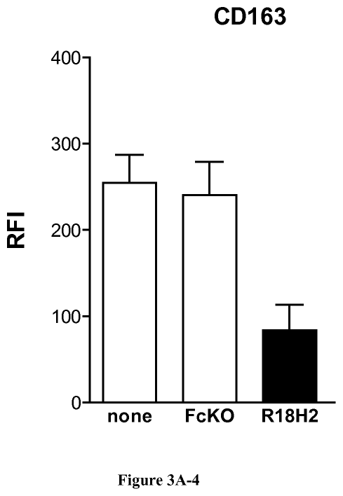

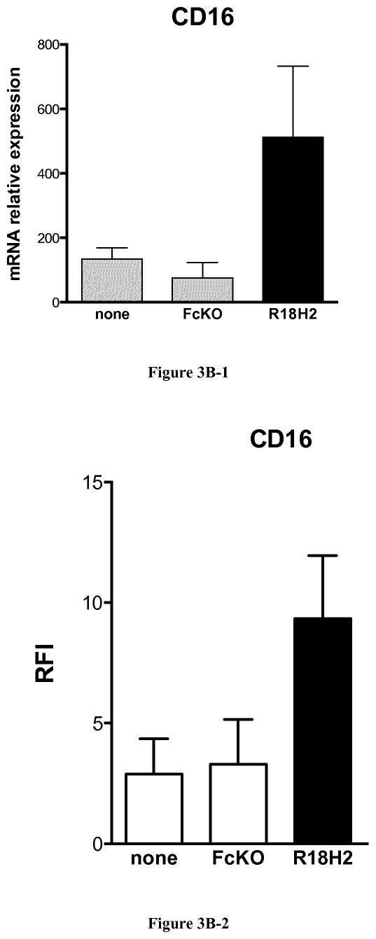

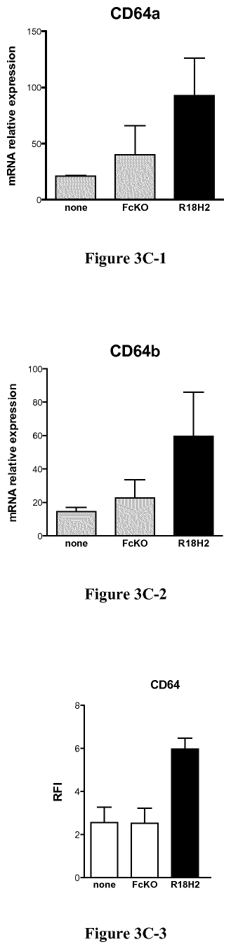

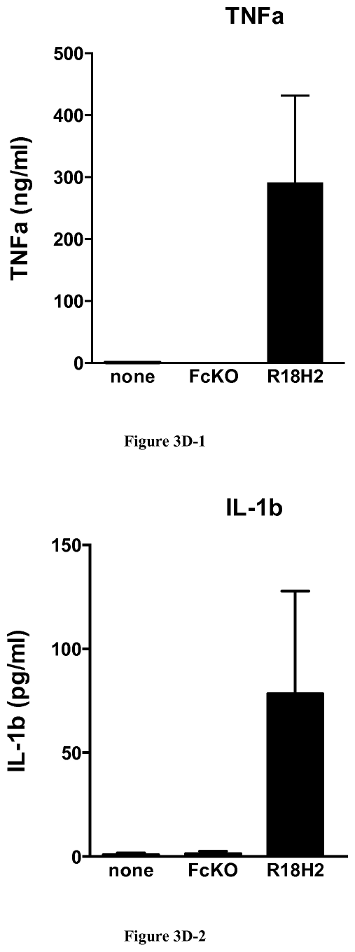

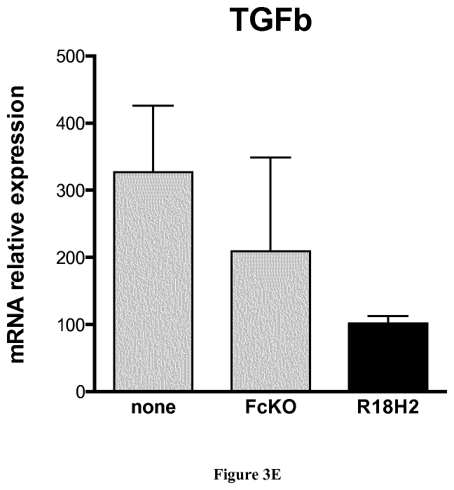

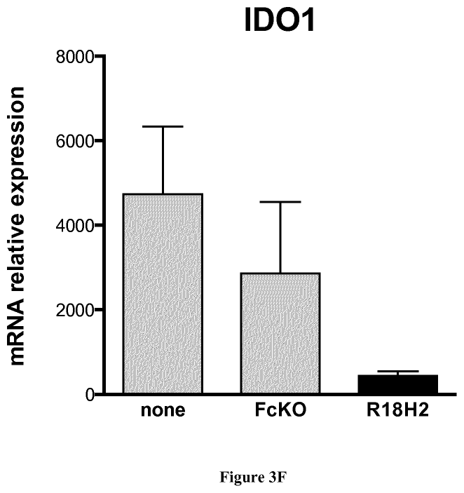

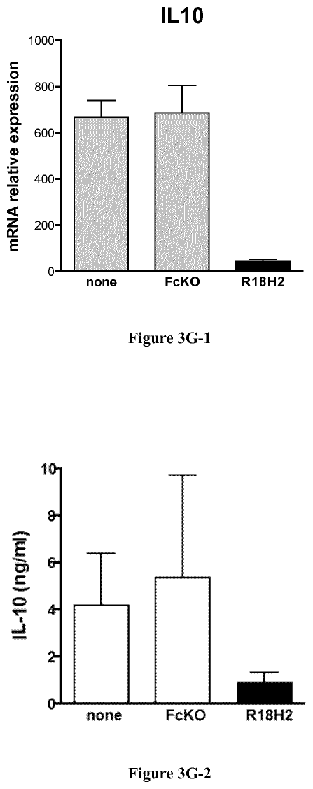

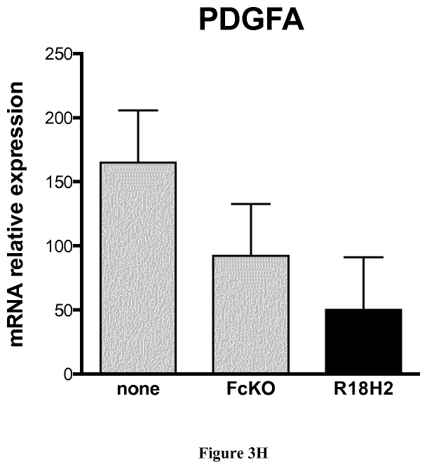

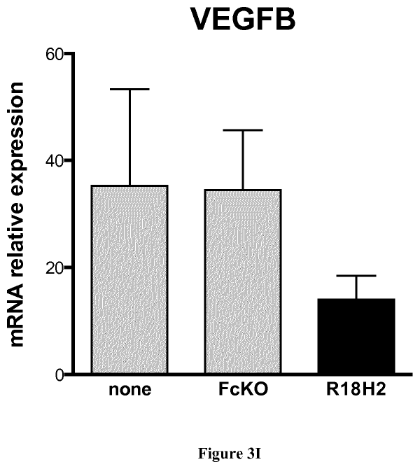

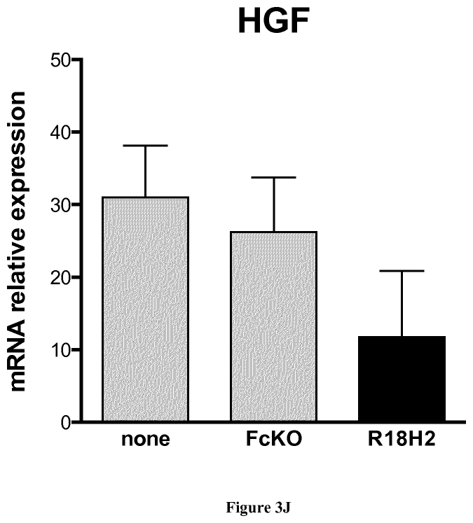

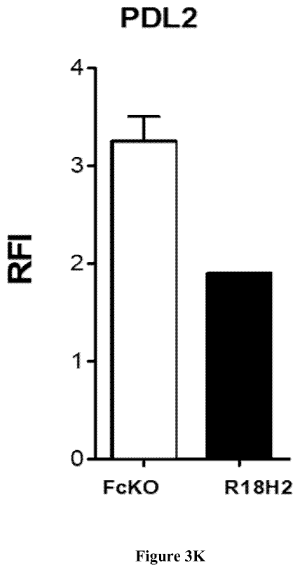

Antibodies adsorbed onto multi-well plates stimulated differentially M2 type macrophages, depending on their potentiality to bind to Fcγ receptors of macrophages. Globally, when M2 type macrophages were cultured in wells without any antibody, no significant variation of markers were observed. With FcKO antibody, only minor variation were observed, corresponding to non-specific binding of those proteins to macrophages. On the opposite, when macrophages interacted with low fucosylated R18H2 antibody, a clear decrease of certain classical markers of M2 type macrophages, such as Sepp1, Stab1, FOLFR2 and CD163, decreased after 3 days of incubation, as shown in FIG. 3A. These variation strongly suggest that type of macrophages could change under stimulation by low fucosylated antibody. These variations were accompanied by an increase of Fcg receptors which bind to antibodies and are involved in ADCC and phagocytosis like CD16 (FIG. 3B) and CD64 (FIG. 3C).

Interestingly, profile of cytokines and soluble peptides detected in culture medium of M2 macrophages after 3 days with low fucosylated R18H2 antibody revealed a clear increase of pro-inflamatory factors usually expressed by M1 macrophages, such as TNFα, IL1β (FIG. 3D) or IL6 (data not shown). Moreover, a decrease of immunosuppressing factors like TGFβ, IDO1 and IL10 (FIG. 3E, FIG. 3F, FIG. 3G) and of pro-angiogenic factors like PDGFα, VEGFβ and HGF was also observed (FIG. 3H, FIG. 3I, FIG. 3J).

Moreover, the decrease in PDL2 expression at the surface of M2 macrophages upon stimulation with the low fucosylated R18H2 antibody (as shown in FIG. 3K) indicates, along with the IL10 decrease, a trend towards less immunosuppressive activity of these phenotype modified macrophages.

All together, these results showed that binding of low fucose antibodies to M2 macrophages, led to shift to an intermediate macrophage phenotype, between M2 and M1, and to variations of several factors conducting to undirect antitumor effects via an inhibition of angiogenesis and a stimulation of immune system.

### Example 6: 3C23K Antibody Blocks Immunosuppression, which Leads to an Activation of the Immune System

A. Materials and Methods

Preparation of Human Monocyte-Derived Macrophages (MDMs)

Peripheral blood mononuclear cells (PBMCs) were obtained from healthy blood donors (Etablissement Frangais de Sang, EFS).

Human Monocytes were isolated from PBMCs using negative selection Monocyte Isolation Kit II (Macs Miltenyi), as recommended by the manufacturer's protocol. Monocytes were cultured at 37° C. and 5% C02 in Macrophage-SFM (Gibco) supplemented with L-glutamine (Invitrogen) and penicillin/streptomycin (PS, Invitrogen).

Isolated monocytes were kept undifferentiated (NS, non-stimulated) or differentiated to anti-tumoral (M1-like) or pro-tumoral macrophages (TMA-like) over three days by stimulating with IFN-γ (Macs Miltenyi, 100 UI/ml)+LPS (100 ng/ml, Sigma) or M-CSF (Macs Miltenyi, 200 UI/ml)+IL-10 (Macs Miltenyi, 50 UI/ml), respectively.

Antibody-Dependent Cell-Mediated Cytotoxicity (ADCC) Assay

SKOV-R2+ cells were pretreated at 4° C. with 10 g/mL anti-AMHRII antibodies: GM102 (also named 3C23K-YB20), 3C23K-CHO or 3C23K-FcKO. Target SKOV-R2+ cells were loaded with BATDA (Bis-acetoxymethyl-2,2′: 6′, 2″-terpyridine-6,6″-dicarboxylate), resuspended in DMEM (Gibco), supplemented with L-glutamine, PS, and 10% heat-inactivated FCS, and added in the effector cells (human macrophages) at 1:1 ratio, at 37° C. for 4 h.

ADCC was measured by using the DELFIA EuTDA-based cytotoxicity assay (PerkinElmer). After 4 h of incubation between target and effector cells, supernatant were incubated with Eu3+ solution, and fluorescence was measured (Envision, PerkinElmer). Data were normalized to maximal (target cells with Triton) and minimal (effector cells alone) lysis and fit to a sigmoidal dose-response model.

Evaluation by Flow Cytometry of Cytotoxic Effects of Macrophages+Antibodies on Ovarian Carcinoma Tumor Cell Line (SKOV-R2+ Cells)

SKOV-R2+ cells

were stained with the CellTrace™ Violet Cell Proliferation kit (Molecular Probes™, Life technology), resuspended in Dulbecco's modified Eagle's medium (DMEM, Gibco), supplemented with L-glutamine, PS, and 10% heat-inactivated fetal calf serum (FCS, Sigma), and added to each type of human macrophages at 1:1 ratio in the presence of each of the 3 anti-AMHRII antibody.

To evaluate SKOV-R2+ cell number and proliferation, the pre-treated or untreated human macrophages were challenged with tumor cells for 3, 4 and 5 days. SKOV-R2+ cell number was calculated by detecting fluorescently labeled cells and their proliferation was evaluated by CellTrace dilution.

A population of 10 000 cells was analyzed for each data point. All analyses were done in a BD Fortessa flow cytometer with Diva software, except CellTrace dilution analyzed by using the Modfit software.

Evaluation of Macrophage Differentiation by Detection of Receptors Expression on Human Macrophages

Receptor expression (CD11b, CD163, CD36, CD206, CD14, CD16, CD32, CD64, CD80, CD282) was evaluated, by flow cytometry, in the membrane of human macrophages after (i) differentiation, and after (ii) 3 days of co-culture between differentiated human macrophages and SKOV-R2+ tumor cells (treated with the different anti-AMHRII antibodies).

Receptors were detected using Cd11b-FITC, CD163-PE, CD36-PE, CD206-APC, CD16-VioBright 515, CD64-PerCP-Vio700, CD80-PE, CD32-PE-Vio770, CD282 (TLR2)-APC, CD14-APC-Vio770 (Miltenyi) and were compared with an appropriate isotype control.

A population of 10 000 cells was analyzed for each data point. The dead cells (positive cells) have been removed from the analysis after labeling with Viability Fixable Dye (Miltenyi). Analyses were gated on CD14 or Cd11b positive cells. All analyses were done in a BD Fortessa flow cytometer with Diva software.

Th1/Th2 T-CD4 Polarization and T-CD8 Activation

Human T cells were isolated from PBMCs using negative selection Pan T Cell Isolation Kit (Macs Miltenyi), as recommended by the manufacturer's protocol. After isolation, cells were stained with the CellTrace™ Violet Cell Proliferation kit (Molecular Probes™, Life technology), resuspended in RPMI 1640 Medium (Gibco), supplemented with L-glutamine, PS, and 10% heat-inactivated FCS, and added in the co-culture of human macrophages+SKOV-R2+ tumor cells (treated with the different anti-AMHRII antibodies mentioned above), at 1:8 ratio for 4 days.

To evaluate Th1/Th2 T-CD4 polarization, T cells were labeled with CD183 (CD183 (CXCR3)-APC, Miltenyi) and analyses were gated on CD4 positive cells (CD4-VioBright FITC, Miltenyi).

To evaluate T-CD8 activation, T cells were labeled with CD183 (CD183 (CXCR3)-APC, Miltenyi) and CD25 (CD25-PE, Miltenyi) and analyses were gated on CD8 positive cells (CD8-PE-Vio770, Miltenyi).

T-CD4 and T-CD8 cell proliferation was evaluated by CellTrace dilution and analyses were gated on CD4 positive cells or CD8 positive cells.

A population of 10 000 cells was analyzed for each data point. All analyses were done in a BD Fortessa flow cytometer with Diva software, except CellTrace dilution analyzed by using the Modfit software.

Production of Cytokines and Chemokines

Cytokines (IL-10, IL-2, IL-6, IL-10, IL-12, IL-23, TNF-α and TGF-β) and chemokines (CCL2, CCL4, CCL5, CXCL9 and CXCL10) release was quantified in the supernatant (i) of differentiated human macrophages, (ii) after 3 days of co-culture between differentiated human macrophages and SKOV-R2+ tumor cells (treated with the different anti-AMHRII antibodies), and (iii) after 4 additive days of this co-culture+ T cells.

The quantification of cytokines and chemokines releases was measured by AlphaLisa immunoassays, according to the manufacturer's instructions (AlphaLisa kit, PerkinElmer).

B. Results

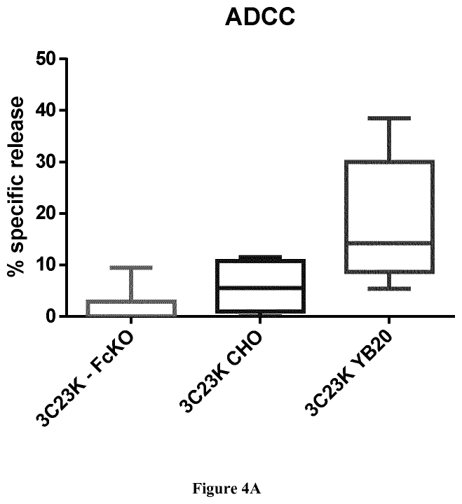

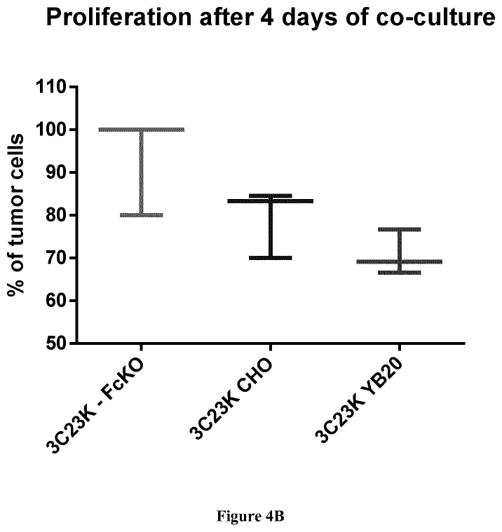

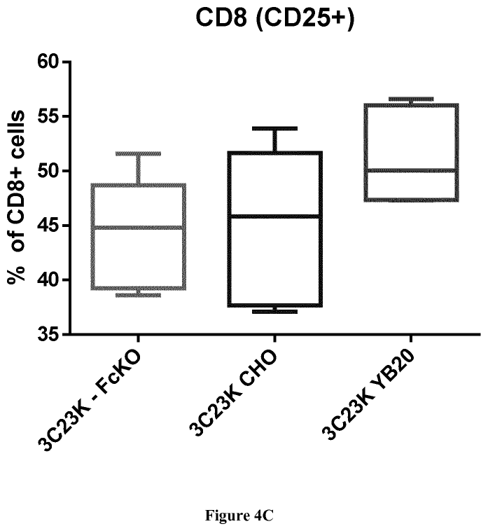

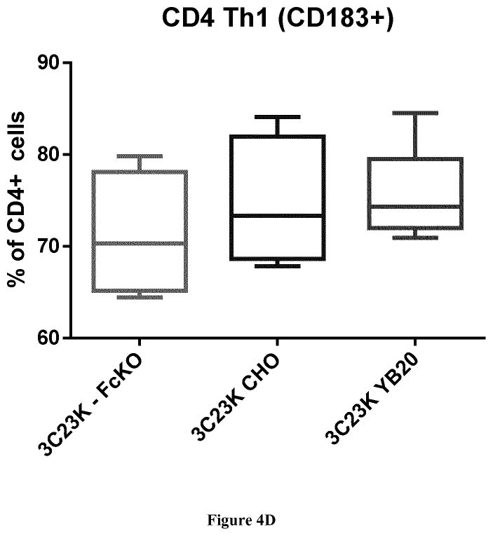

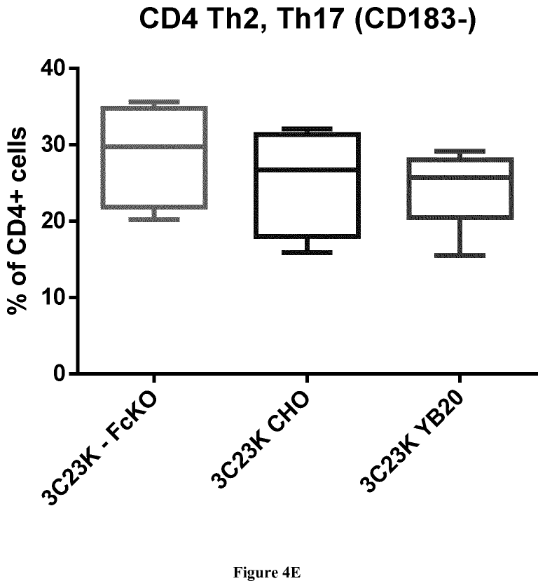

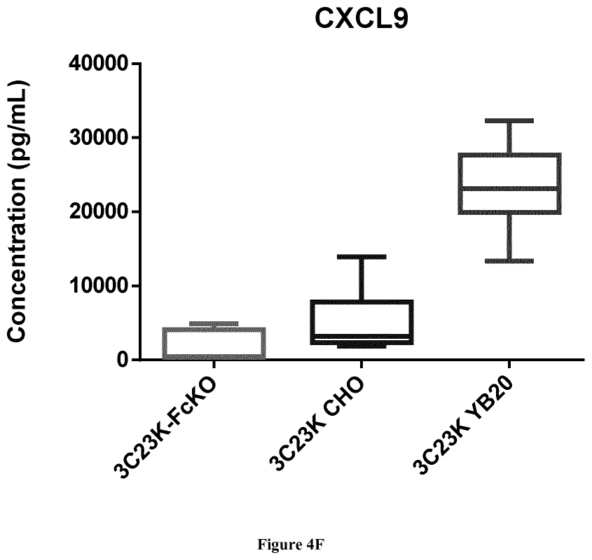

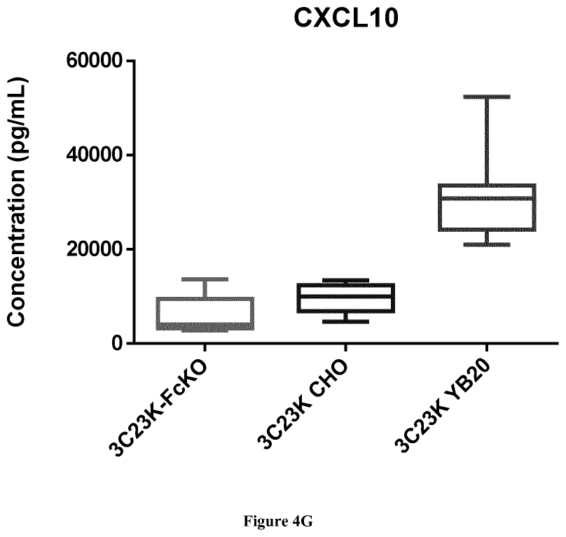

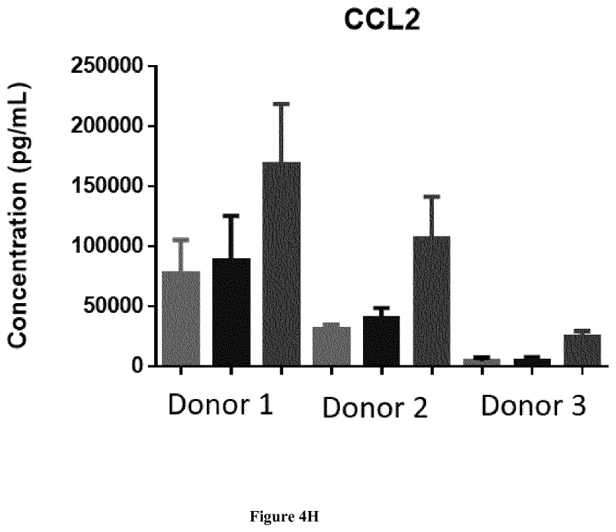

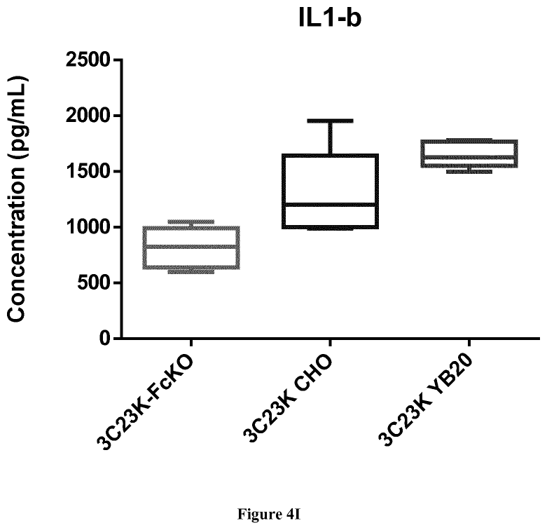

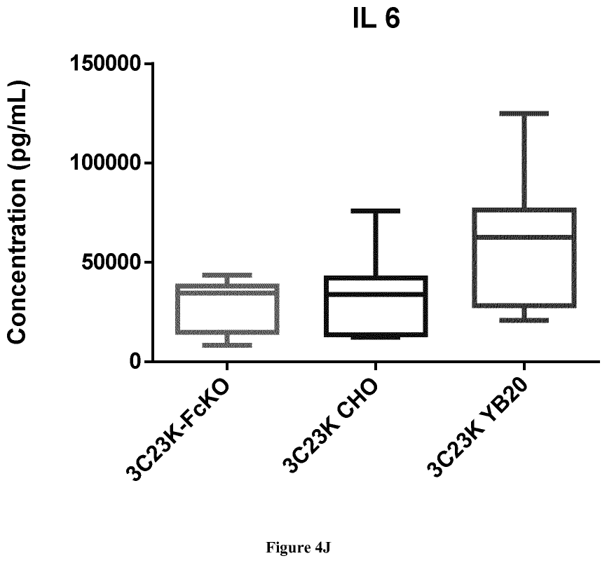

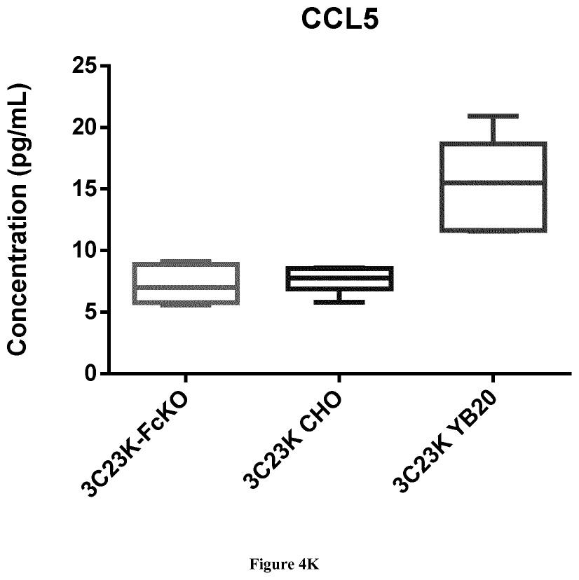

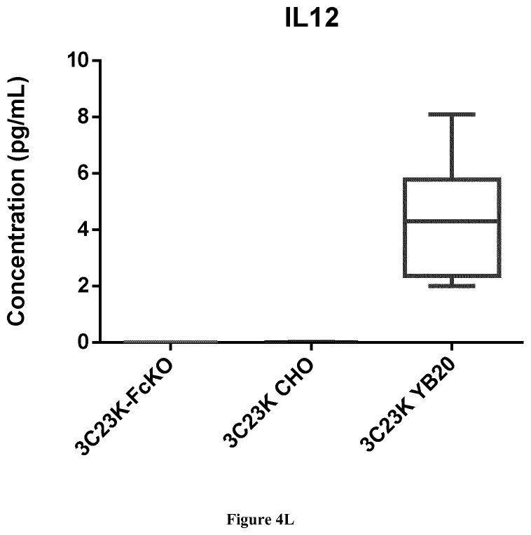

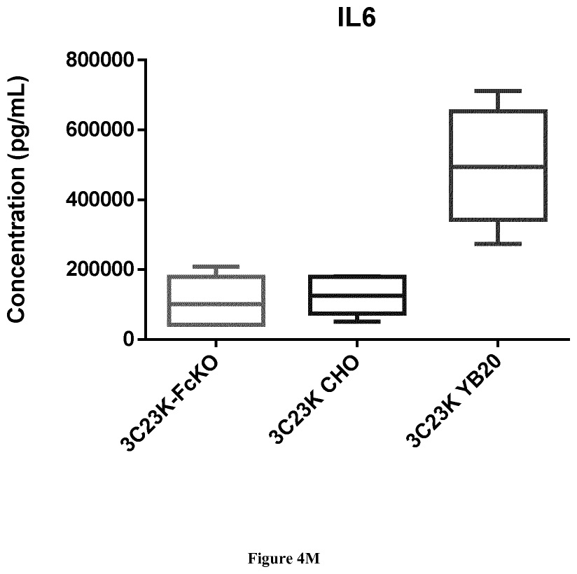

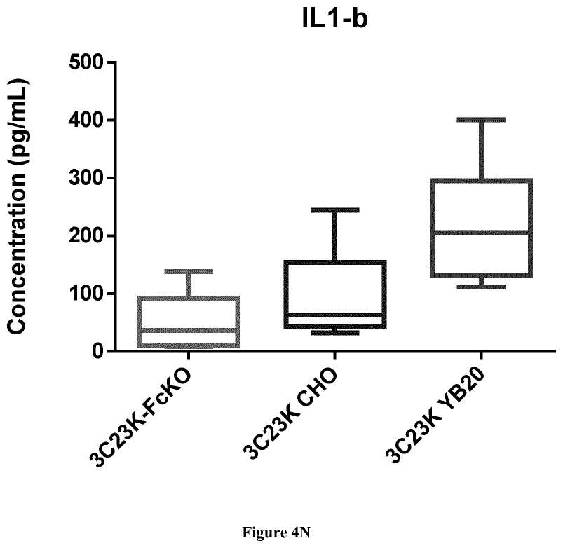

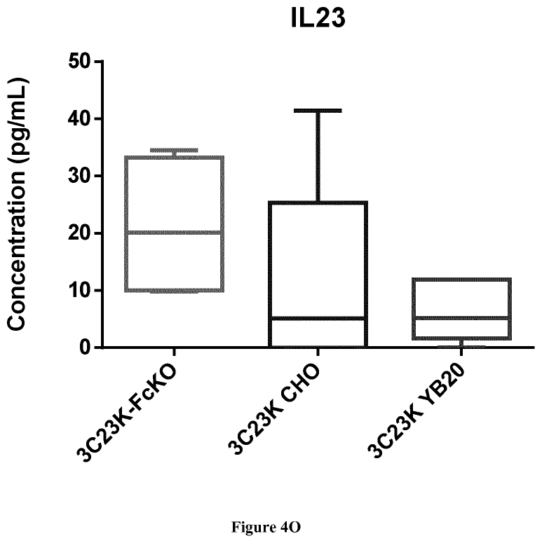

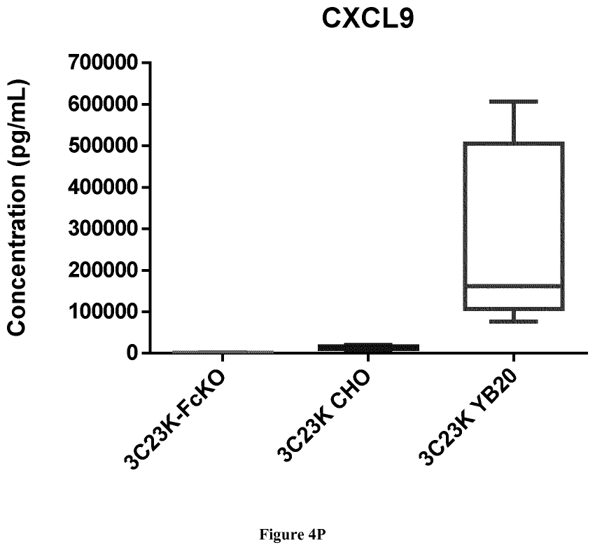

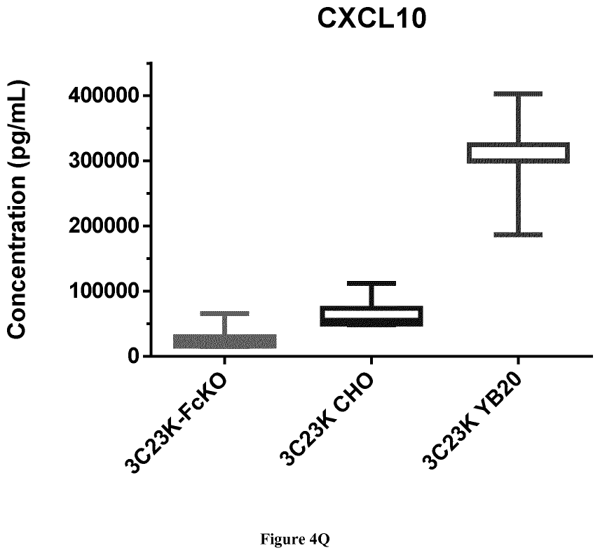

All experiments were performed with PBMC from three different and independent healthy donors. ADCC measured in the presence of macrophages undifferentiated or differentiated in TAM-like (with addition of M-CSF and IL-10) was found clearly higher with 3C23K-YB20 low fucose antibody in comparison to 3C23K-CHO or 3C23K-FcKO, used as inactive control. Data with TAM-like are presented in FIG. 4A. This cytolytic activity could explain, at least partially, the decrease of tumor cells after four days of co-incubation with TAM-like macrophages. This decrease was also higher with 3C23K YB20 than with 3C23K-CHO, as shown in FIG. 4B.

When T cells were added to co-culture of TAM-like macrophages and tumor cells, an increase of percentage of memory CD8+ lymphocytes was observed (FIG. 4C). A tendency of increase of Th1 CD4 regulatory T-cells was also found under the same experimental conditions (FIG. 4D), in parallel to a decrease of Th2 CD4+ T-cells. (FIG. 4E) All these modulations are in accordance with an increase of long-term T-cells-mediated antitumor activity. All these variations were higher with 3C23K-YB20 than with 3C23K-CHO and 3C23K-FcKO.

Interestingly, profile of cytokines and chemokines detected in medium of co-culture of TAM-like+ tumor cells in the presence of anti-AMHRII 3C23K-YB20 antibody revealed a clear increase of CXCL9 (FIG. 4F) and CXCL10 (FIG. 4G), two factors involved in T-cells recruitment and of CCL2, a factor involved in macrophages recruitment whereas basal level of this last factor was different in each donor (FIG. 4H). Moreover, when T-cells were added in this co-culture, increases of two pro-inflammatory cytokines, IL6 (FIG. 4I) and IL1b (FIG. 4J) and of CCL5 (FIG. 4K), a factor associated with T-cells infiltration, were detected with 3C23K-YB20, also higher than with 3C23K-CHO and 3C23K-FcKO.

All together, these results showed that addition of 3C23K-YB20 to co-culture of tumor cells+TAM-like macrophages then + T-cells, i.e. conditions mimicking pathological situation into the tumor, led to direct tumor cells lysis and activation of antitumor T-cells response. All these observations were higher with 3C23K-YB20 than the other anti-AMHRII antibodies tested.

Interestingly, favorable effects of 3C23K-YB20 were not restricted to conditions with TMA-like macrophages. Similar experiments with non-stimulated (NS) macrophages co-cultured with tumor cells and antibodies permitted to show an increase of pro-inflammatory factors such as IL12 (FIG. 4L), IL6 (FIG. 4M) and IL1b (FIG. 4N) in parallel to a decrease of IL23 (FIG. 4O), a pro-tumoral and pro-angionenic cytokine. Moreover, as described above with TAM-like macrophages, an increase of CXCL9 (FIG. 4P) and CXCL10 (FIG. 4Q), two anti-angiogenic chemokines involved in T-cells recruitment, was also observed. All these variations were more significant with 3C23K-YB20 low fucose antibody than with others. These results strongly suggest that 3C23K-YB20 could influence T-cells antitumor activity via undifferentiated macrophages as well as via TAM-like macrophages.

### Example 7: Activation of Macrophages Present in the Tumor Tissue of Cancer Patients Administered with a Glyco-Engineered Antibody

A. Materials and Methods

To identify multiple targets in the same tissue section, TSA-based multiplex immunofluorescence is used in this study. Tyramide Signal Amplification (TSA) is based upon the patented catalyzed reporter deposition (CARD) technique using derivatized tyramide. In the presence of small amounts of hydrogen peroxide, immobilized HRP converts the labeled substrate (tyramide) into a short-lived, extremely reactive intermediate. The activated substrate molecules then very rapidly react with and covalently bind to electron rich regions of adjacent proteins. This binding of the activated tyramide molecules occurs only immediately adjacent to the sites at which the activating HRP enzyme is bound. Multiple deposition of the labeled tyramide occurs in a very short time (generally within 3-10 minutes). Subsequent detection of the label yields an effectively large amplification of signal. The advantage of this technology is that multiple primary antibodies raised in the same species can be detected on the same tissue slide. Each reaction is stopped when the TSA-fluorochrome has precipitated. This can be repeated to reach 5 targets. In our lab, the Ventana Discovery ULTRA automated slide stainer is available to automate the procedure. This instrument allows for efficient, reproducible, walk-away staining of FFPE tissue slides.

In the multiplex application, the following fluorophores disclosed in Table 5 below are used:

| | | DISCOVERY | DISCOVERY | DISCOVERY | DISCOVERY | DISCOVERY |
|---|---|---|---|---|---|---|
| | DAPI | DCC | FAM | Rhod 6G | Red 610 | Cy5 |
| Excitation* | 350/50-nm | 436/20-nm | 490/20-nm | 546/10-nm | 580/25-nm | 640/30-nm |
| Beams | 400-nm | 455-nm | 505-nm | 556-nm | 600-nm | 660-nm |
| Emission* | 460/50-nm | 480/30-nm | 520/50-nm | 572/23-nm | 625/30-nm | 690/50-nm |

These fluorophores, secondary antibody systems and primary antibodies represent the assay specific reagents. All other ancillary reagents used to perform the staining (pretreatment, wash and denaturation buffers) are considered general purpose reagents. The assay limitations are determined by the imaging platform available and used. All whole slide images are produced using a P250 Panoramic scanner from 3DHistech which is equipped with suitable filters to separate the fluorophores used (Rhodamin6G, RED610, DCC, FAM, Cy5). Due to spectral characteristics, the DAPI and DCC signals can however not be separated to date. Therefore, the nuclear counterstain has been left out.

Multiplex immuno fluorescence development was requested for the following markers/purposes:
Cytokeratin (CK), CD3, CD4, CD8, FoxP3 for lymphocytes
CK, CD14, HLADR, CD206 and/or CD163 for macrophages
CK, CD56, CD15, Granzyme, DC lamp for dendritic cells, polymorphonuclear cells, natural killer cells
CK, CD45, CD16, CD32, and CD64 for effector cells versus immune cells

These four multiplex assays were validated with following sequential labeling:

1/ Anti-CD3 clone 2GV6, anti-CD4 clone SP35, anti-CD8 clone C8/144B, anti-FoxP3 clone D2W8E and anti-CK clones cocktail AE1/AE3

2/ anti-CD14 clone EPR3653, anti-CD68 clone KP-1, anti-CD163 clone MRQ-26, anti-MHC-II clone EPR11226 and anti-CK clones cocktail AE1/AE3

3/ anti-CD16 clone SP175, polyclonal anti-Granzyme B, anti-CD8 clone C8/144B and anti-NKp46

4/ anti-CD15 clone MMA, anti-CD64 clone 3D3, polyclonal anti-CD206 and anti-LAMP3 clone 13A205.

B. Results

During the Phase I study of GM102 the presence of several cell types was investigated using multiplex fluorescent staining and analysis on FFPE paired and baseline ovarian carcinoma biopsies. The multiplex staining covered immune infiltrates and the evaluation of monocyte/macrophage differentiation and phagocytic activity. Baseline samples have been biopsied 7 to 15 days before GM102 and second biopsies have been performed after 1.5 month of treatment.

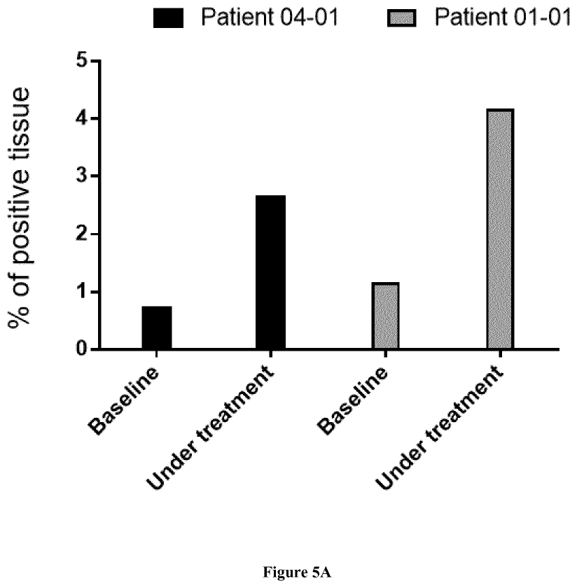

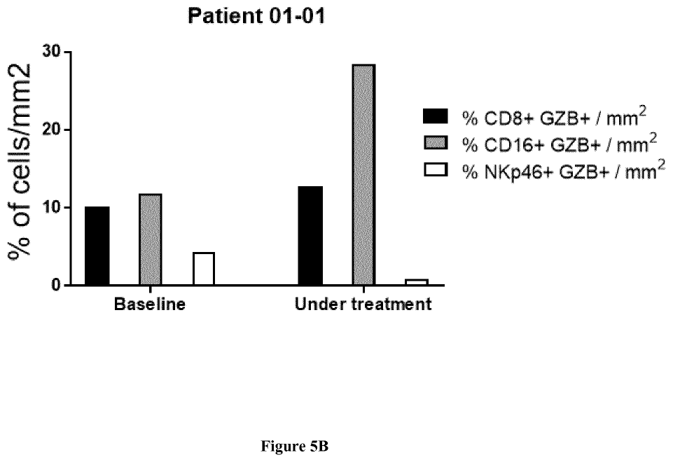

Since only two paired biopsies were studied, the effect of GM102 treatment has only been evaluated in a descriptive way, with no statistical analysis for the observed phenomena. Initial baseline samples were characterized by a variable presence of an immune infiltrate. The most prominent observation of this study is the effect of GM102 on the monocyte-like CD16+ cell population (FIG. 5A), a cell type which was already abundantly present at baseline. Under GM102 treatment, CD16+ stained area increased markedly for the studied patients, which was however not reflected by an increase of CD16+ cell density, as if the CD16+ cells got charged with CD16 upon GM102 treatment. This observation suggests an activation of CD16+ cells, the effector cells (mainly macrophages) involved in antitumor activity of GM102.

In addition, an increase was observed of Granzyme B expression under GM102 treatment (FIG. 5B). Granzyme B is a 29 kDa member of the granule serine protease family stored specifically in NK cells or cytotoxic T cells. Cytolytic T lymphocytes (CTL) and natural killer (NK) cells share the ability to recognize, bind, and lyse specific target cells. They are thought to protect their host by lysing cells bearing on their surface ‘non-self’ antigens, usually peptides or proteins resulting from infection by intracellular pathogens. Granzyme B is crucial for the rapid induction of target cell apoptosis by CTLs in the cell-mediated immune response (Rousalova & Krepela, 2010, Int. J. Oncol. 37: 1361-1378; Vaskoboinik at al., 2015, Nat. rev. Immunol. 15: 388-400). Cytotoxic T lymphocytes (CTL) and natural killer (NK) cells are the major actors in the elimination of neoplastic and virally infected cells. However, in these biopsies natural killer cells, as visualized by NKp46, were only sporadically seen and CD8+ lymphocytes. This observation confirmed in clinical samples that treatment of GM102 induced cytolytic activity of CD8+T lymphocytes.

### Example 8: Activation of NK Cells, Monocytes and ICOS+ T Cells in Cancer Patients Administered with a Glyco-Engineered Antibody

A. Materials and Methods

In phase I study of GM102, sampling of 5 mL blood at 4 timepoints was planned for each patients of escalating cohorts. Timepoints are at Day 1 before first GM102 infusion (named C1J1-SOI) & end of first GM102 infusion (named C1J1-EO1), at day 15, before second GM102 infusion (C1J15-SOI) and at steady-state, e.i. at day 57, the end of second 28-day cycle (C3J1-SOI).

For an scientific exploration purpose, several different markers were monitored at each clinical site of this study.

At Gustave Roussy, five patients were included. The LIO (Laboratoire d'Immunomonitoring en Oncologie) received a total of 15 samples as detailed in the table 6 below.

TABLE 6

| | | | | Number of | Cycle 1 | | | Cycle 3 |
|---|---|---|---|---|---|---|---|---|
| | | Inclusion | Graph | received | | C1J1 | C1J15 | C3J1 |
| Cohorts | Center | N° | N° | samples | C1J1 (SOI) | (EOI) | (SOI) | (SOI) |
| 7 mg/ml | GRCC | 01-01 | 001 | 3 | Dec. 8, 2016 | Dec. 8, | Dec. 22, | |
| q2w | | | | | | 2016 | 2016 | |
| | GRCC | 01-02 | 002 | 4 | Dec. 1, 2016 | Dec. 1, | Dec. 15, | Feb. 10, |
| | | | | | | 2016 | 2016 | 2017* |
| 10 mg/ml | GRCC | 01-03 | 003 | 3 | Feb. 6, 2017 | Feb. 6, | Feb. 20, | |
| q2w | | | | | | 2017 | 2017 | |
| 20 mg/ml | GRCC | 01-04 | 004 | 1 | Mar. 27, 2017 | | | |
| q2w | GRCC | 01-05 | 005 | 4 | May 29, 2017 | May 30, | Jun. 12, | Jul. 24, |
| | | | | | | 2017** | 2017 | 2017 |

SOI: Start Of Intusion;
EOI: End Of Infusion
*Sample taken at EOI on Feb. 9, 2017 and received the day after at the LIO;
**Sample taken at EOI on May 29, 2017 and received the day after at the LIO.

All received samples were analyzed. PBMCs were isolated from all samples and are stored in a box dedicated to Gamamabs—GM102 study in liquid nitrogen tank with restricted access to authorized personal. All materials used were detailed in tables 7, 8 and 9 below:

TABLE 7

| Equipment | Specification | Catalog No. | Supplier |
|---|---|---|---|
| Centrifuge | Heraeus Multifuge X3R | 75004518 | Thermo Fisher |
| | | | Scientific |
| 100-1000 | Monochanel Research Pipette, | 4701070 | Eppendorf |
| μL pipete | 100-1000 μL, variable, manual | | |
| 5-20 μL | Monochanel Research Pipette, | 4701070 | Eppendorf |
| pipete | 5-20 μL, variable, manual | | |
| Biosafety | Class II microbiological safety | 51025411 | Thermo Fisher |
| cabinet | station | | Scientific |

TABLE 8

| Material | Specification | Catalog No. | Supplier |
|---|---|---|---|
| 50 mL tubes | Volume up to 50 mL, sterile, | 191050 | Dutcher |
| | PP | | |
| 15 mL tubes | Volume up to 15 mL, sterile, | 171015 | Dutcher |
| | PP | | |
| pipetes 5 mL | Serological, pipeting volume | 86.1253.001 | SARSTEDT |
| | 1-5 mL | | |
| pipetes 10 mL | Serological, pipeting volume | 86.1254.001 | SARSTEDT |
| | 1-10 mL | | |
| pipetes 25 mL | Serological, pipeting volume | 86.1685.001 | SARSTEDT |
| | 2-25 mL | | |
| Pipete tips | Filtertips, pipeting volume | | |
| | 100-1000 μL, sterile | | |
| Pipete tips | Filtertips, pipeting volume | | |
| | 10 μL, sterile | | |
| Cryotube | Volume 1.8 ml | 479-6843 | VWR |

TABLE 9

| Reagent | Specification | Catalog No. | Supplier | Storage/validity |
|---|---|---|---|---|
| Ficoll | Ficoll Histopaque | 10771-500 mL | SIGMA | In dark +4° C. |
| | 1077 | | | |
| PBS | PBS pH 7.4 (10×), | 700011-036 | GIBCO | 4-30° C., 36 |
| | 500 mL, no Mg, | | | months |
| | no Ca | | | |
| Hyclone | HyCryo-2x- | SR30001.02 | Thermo | In dark +4° C. |
| | Cryopreservation | | Fisher | |
| | Media | | | |

PBMCs were isolated according to the following procedure:
1. Disinfect the tube of blood by wiping it with Anios.
2. Take 50 mL tubes prefilled with 15 mL of Ficoll and slowly layer 35 mL of the diluted blood over the Ficoll being careful not to mix—two layers should be clearly separated. (For other volumes keep the ratio diluted blood:Ficoll around 2/3).
3. Securely close the tube and centrifuge at 400 g for 20 min at room temperature (RT), BREAK OFF.
4. Use a sterile single use 10 mL pipet to recuperate the mononuclear cells layer (ring) and transfer in a new 50 mL tube. (Optionally: the upper phase can be discarded before recuperating the ring).
5. Add PBS up to 50 mL and centrifuge at 800 RPM for 15 min at 15° C.
6. Immediately flick out supernatant using pipet and stop at 3 mL before the bottom.
7. Flick to resuspend pellet.
8. Add 50 ml of PBS over the remaining cell pellet and mix to thoroughly resuspend.
9. Centrifuge at 300 g for 10 min at 15° C.
10. Flip out the tube and add 1 or 2 mL of PBS to count the cells.

PBMCs were stored according to the following procedure:
1. Arrange 90 μL of Blue Hayem in a 96-well plate and add 10 μL of the previously resuspended pellet to count only PBMC.
2. Arrange 90 μL of Blue Trypan in a 96-well plate and add 10 μL of the previously resuspended pellet to count only living cells.
3. Count the cells on Malassez's slide and freeze between 5 and 10 million living cells in Hyclone (1 mL per cryotube).
4. Put the cryotubes in a “Mr Freeze” box and put it at −80° C. for at least 24 hours before transferring them to the nitrogen tank in Gamamabs—GM102's box.

Blood Immune Phenotyping was performed by flow cytometry with the following procedure and markers described in tables 10 to 17 below.

TABLE 10

| Equipment | Specification | Catalog No. | Supplier |
|---|---|---|---|
| Centrifuge | Heraeus Multifuge X3R | 75004518 | Thermo Fisher |
| | | | Scientific |
| 100-1000 | Monochanel Research Pipette, | 4701070 | Eppendorf |
| μL pipete | 100-1000 μL, variable, manual | | |
| 2-5 μL | Monochanel Research Pipette, | 4701070 | Eppendorf |
| pipete | 2-5 μL, variable, manual | | |
| 5-20 μL | Monochanel Research Pipette, | 4701070 | Eppendorf |
| pipete | 5-20 μL, variable, manual | | |
| Biosafety | Class II microbiological safety | 51025411 | Thermo Fisher |
| cabinet | station | | Scientific |
| Flow | Gallios 561 Ready 10 colors 3 | A94303 | Beckman |
| cytometer | lasers | | |

TABLE 11

| Material | Specification | Catalog No. | Supplier |
|---|---|---|---|
| Blue facs tube | Sample Tubes for FC 500 | 2523749 | Beckman |
| | and EPICS ™ XL ™ Flow | | |
| | Cytometers | | |
| 50 mL tubes | Volume up to 50 mL, sterile, | 191050 | Dutcher |
| | PP | | |
| 15 mL tubes | Volume up to 15 mL, sterile, | 171015 | Dutcher |
| | PP | | |
| Pipetes 5 mL | Serological, pipeting volume | 86.1253.001 | SARSTEDT |
| | 1-5 mL | | |
| Pipetes 10 mL | Serological, pipeting volume | 86.1254.001 | SARSTEDT |
| | 1-10 mL | | |
| Pipete tips | Filtertips, pipeting volume | S1111-6700 | Star Lab |
| | 100-1000 μL, sterile | | |
| Pipete tips | Filtertips, pipeting volume | S1111-3700 | Star Lab |
| | 10 μL, sterile | | |
| Pipete tips | Filtertips, pipeting volume | S1111-1700 | Star Lab |
| | 200 μL, sterile | | |
| DURACLONE | TUBE PANEL TSCM | B57792 | Beckman |

TABLE 12

| Reagent | Specification | Catalog No. | Supplier | Storage/validity |
|---|---|---|---|---|
| Fixative | Fixative solution | A07800 | Beckman | In dark +4° C. |
| solution | 10× | | coulter | |
| PBS | PBS pH 7.4 (10×), | 700011-036 | GIBCO | 4-30° C., 36 |
| | 500 mL, no Mg, | | | months |
| | no Ca | | | |
| Beads | Flow-count | 7547053 | Beckman | In dark +4° C. |
| | Fluorospheres | | coulter | |
| VersaLyse | Lysing Solution | A09777 | Beckman | 18-25° C. |
| | | | coulter | |

TABLE 13
Duraclone Panel:

| | FITC | PE | ECD | PC5.5 | PC7 | APC | AA700 | AA750 | PB | Kro |
|---|---|---|---|---|---|---|---|---|---|---|
| TSCM | CD95 | CD197 | HLA-DR | CD25 | CD45RA | CD278 | CD3 | CD127 | CD4 | CD8 |

TABLE 14
Liquid formulation Panels:

| | FITC | PE | ECD | PC5.5 | PC7 | APC | AA700 | AA750 | BV421 | KrO |
|---|---|---|---|---|---|---|---|---|---|---|
| NK-NCR | CD335 | CD336 | HLA-DR | CD337 | CD137 | CD314 | CD3 | CD16 | CD56 | CD8 |

TABLE 15

| | FITC | PE | ECD | PC5.5 | PC7 | APC | AA700 | AA750 | PB | BV510 |
|---|---|---|---|---|---|---|---|---|---|---|
| FcγR- | CD32 | CD54 | CD69 | CD244 | CD64 | CD163 | CD14 | CD16 | CD15 | CD56 |
| Activation | | | | | | | | | | |

TABLE 16

| Antibodies in | | | | |
|---|---|---|---|---|
| liquid | | | | |
| formulation | Supplier | Catalog No. | Clone No. | Emplacement |
| CD3-AA700 | Beckman | B10823 | UCHT1 | Laboratory |
| CD8-KrO | Beckman | B00067 | B9.11 | refrigerator |
| CD14-AA700 | Beckman | A99020 | RMO52 | LIO |
| CD15-PB | Beckman | A74775 | 80H5 | part 505 |
| CD16-AA750 | Beckman | A66330 | 3G8 | |
| CD32-FITC | AbD Serotec | MCA1075F | AT10 | |
| CD54-PE | Beckman | IM1239U | 84H10 | |
| CD56-BV421 | Ozyme | BLE318328 | HCD56 | |
| CD64-PC7 | Beckman | B06025 | 22 | |
| CD69-ECD | Beckman | 6607110 | TP1.55.3 | |
| CD137-PC7 | Ozyme | BLE309818 | 4134-1 | |
| CD163-APC | Miltenyi | 130-112-129 | REA812 | |
| CD244-PC5.5 | Beckman | B21171 | C1.7 | |
| CD314-APC | Beckman | A22329 | ON72 | |
| CD335-FITC | Ozyme | BLE331923 | 9E2 | |
| CD336-PE | Beckman | IM3710 | Z231 | |
| HLA-DR-ECD | Beckman | IM3636 | Immu-357 | |

TABLE 17

| Duraclone tube (Num and TSCM | Liquid antibodies (Senescence |
|---|---|
| panels) | Panel) |
| Take out the duraclone tube | Distribute panel antibodies in a blue |
| | facs tube |

Add 100 μL of blood in and vortex for 10 seconds (inside the biosafety
cabinet)

| | |
|---|---|
| Incubate 20 minutes at Room | Incubate 15 minutes at Room |
| Temperature in the dark | Temperature in the dark |

Prepare the lysis solution: 1 mL Versalyse for 25 μL of Fixative Solution

| | |
|---|---|
| Add 2 mL of lysis solution | Add 1 mL of lysis solution |

Vortex 10 seconds and incubate 20 min at Room Temperature in the dark

| | |
|---|---|
| Add 2 mL of 1 × PBS | Add 3 mL of 1 × PBS |

Centrifuge 5 min 300 g
Eliminate the supernatant by inversion
Add 3 mL of 1 × PBS and vortex
Centrifuge 5 min 300 g
Eliminate the supernatant by inversion
Suspend the pellet in 250 μL of PBS1X
Acquire the tube with Gallios Cytometer

B. Results

Each marker listed above was measured and analyzed along the treatment. On the 5 patients tested, no significant variation was identified for the main immune populations of circulating cells (Ncells, Monocytes, Neutrophils, Eosinophils and T cells CD4+, CD8+ and Treg).

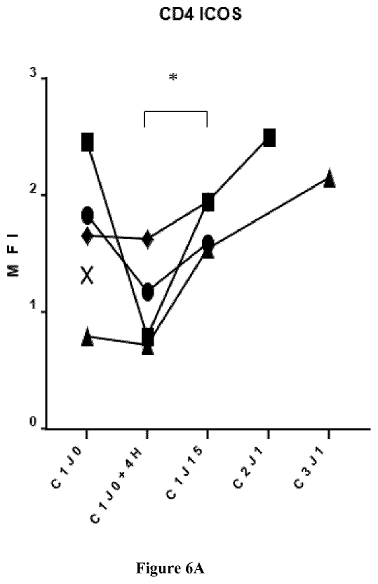

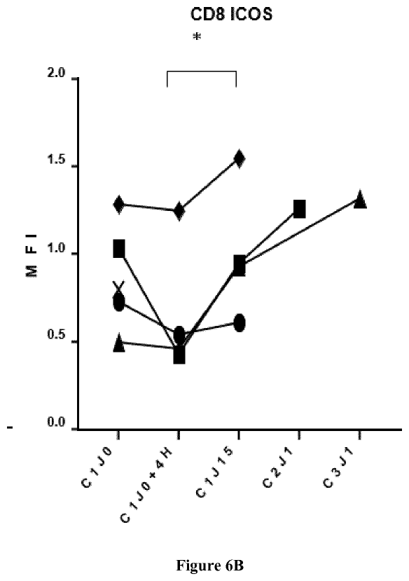

Some markers suggested activation of monocytes and NK cells. A significant increase of CD16 expression was observed on NK cells between C1J1-EO1 and C1J15, after a decrease during injection of GM102 (fig: 6A). A statically non-significant tendency of increase was also observed with CD69 expression on monocytes (FIG. 6B). Whereas its immunoregulatory role remains unclear, CD69 expression is known to increase following immune cells activation (Sancho et al., 2005, Trends in Immunology, Vol. 26 (3): 137-140). These increases could be translated as signs of activation of monocytes and NK cells.

Interestingly, the major and significant variation was an increase of ICOS expression on T cells between C1J1-EOI and C1J15 (fig: 6C). ICOS is a receptor involved in T cell activation (Yao et al., 2013, Nature Reviews, Vol. 12: 130-146; Mahoney et al., 2015, Nature Reviews, Vol. 14: 561-584) and it is known as a pharmacodynamic marker of ipilumumab, an anti-CTLA4 antibody, inhibitor of immunologic checkpoint (Tang et al., 2013, American association for cancer Research Journal, Vol. 1(4): 229-234). Therefore this increase confirms in patients that GM102 can reverse immunosuppression.

### Example 9: Effect of GM102 on Circulating Monocytes

A. Materials and Methods

In phase I study of GM102, sampling of 5 mL blood at 4 timepoints was planned for each patients of escalating cohorts. Timepoints are at Day 1 before first GM102 infusion (named C1J1-SOI) & end of first GM102 infusion (named C1J1-EO1), at day 15, before second GM102 infusion (C1J15-SOI) and at steady-state, e.i. at day 57, the end of second 28-day cycle (C3J1-SOI).

For an scientific exploration purpose, several different markers were monitored at each clinical site of this study.

Human PBMC were isolated from the blood by a density gradient centrifugation method on Lymphoprep (Abcys). For lymphocyte population infiltration and their activation, the PBMC were labeled with the following antibodies: CD45-VioGreen, CD3-VioBlue, CD4-APCVio770, CD8-PerCP, CD25-PE, CD56-APC, CD19-PEVio770 and CD69-FITC (Myltenyi Biotec).

For blood monocytes, classical, intermediate and non-classical populations were evaluated with the following antibodies: CD45-VioGreen, CD16-PE and CD14-PerCPVio700 (Myltenyi Biotec). Appropriate fluorochrome-matched isotype antibodies were used to determine nonspecific background staining. All staining were performed on 100 μL of PBS−/− 1% heat-inactivated fetal calf serum. A population of 10.000 cells was analyzed for each data point. All analyses were done in a BD Fortessa flow cytometer with Diva software.

B. Results

Percentages of T cells, NK cells and monocytes before the first infusion of 3C23K were found variable between patients, indicating various immunocompetency between patients. Under and after treatment, no notable variation was observed in T cells and NK cells populations. On the opposite, variations were observed with monocytes subsets.

Human blood monocytes are heterogeneous and conventionally subdivided into three subsets based on CD14 and CD16 expression. «Classical monocytes» (CD14high CD16-) represent 90-95% of total monocytes in healthy donors, whereas «Non-classical» (CD14low CD16+) and «Intermediate» (CD14high CD16+) populations are less represented (5-10%).

In 3 out of 4 patients with ovarian adenocarcinoma, the proportion of “classical monocytes” was strongly decreased in patient before the first infusion (mean value=37.5%) compared to healthy patient. This phenomenon is usually observed in patients with ovarian cancer. Consequently, the proportion of intermediate monocytes before the first infusion was increased in patient with ovarian adenocarcinoma (mean value=46.5%) compared to healthy donors.

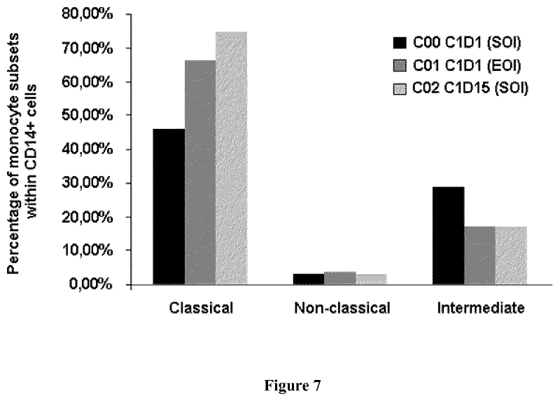

Interestingly, percentage measure under and after treatment with 3C23K revealed a large increase of classical monocyte subset accompanied by a decrease of the proportion of intermediate monocyte subset in patients (with means values of 54.6% and 30.5% respectively), as exemplified with patient 04-06 in FIG. 7. Such variations of monocyte subsets were also observed in blood samples of the 4 patients studied under the same protocol at Institut Bordet. These variations are signs of modification of monocyte phenotypes. Variations of monocyte subpopulations were recently described in response to immune check-point inhibitors (Krieg C., Nowicka M., Guglietta S., Schindler S., Hartmann F. J., Weber L. M., et al. (2018). High-dimensional single-cell analysis predicts response to anti-PD-1 immunotherapy. Nat Med. 24, 144-153), leading to activation of lymphocytes (by blockage of inhibitory signals). Our data show that 3C23K, without any binding to immune check-point, can also modify proportions of monocytes subsets that could, consequently, activate lymphocytes.

TABLE 3
Sequences

| SEQ ID NO. | Type | Description |
|---|---|---|
| 1 | Nucleic acid | 3C_23 VL without leader |
| 2 | Peptide | 3C_23 VL without leader |
| 3 | Nucleic acid | 3C_23 VH without leader |
| 4 | Peptide | 3C_23 VH without leader |
| 5 | Nucleic acid | 3C_23K VL without leader |
| 6 | Peptide | 3C_23K VL without leader |
| 7 | Nucleic acid | 3C_23K VH without leader |
| 8 | Peptide | 3C_23K VH without leader |
| 9 | Nucleic acid | 3C_23 light chain without leader |
| 10 | Peptide | 3C_23 light chain without leader |
| 11 | Nucleic acid | 3C_23 heavy chain without leader |
| 12 | Peptide | 3C_23 heavy chain without leader |
| 13 | Nucleic acid | 3C_23K light chain without leader |
| 14 | Peptide | 3C_23K light chain without leader |
| 15 | Nucleic acid | 3C_23K heavy chain without leader |
| 16 | Peptide | 3C_23K heavy chain without leader |
| 17 | Peptide | 3C23K/3C23 |
| 18 | Peptide | 3C23KR/6B78 |
| 19 | Peptide | 5B42 |
| 20 | Peptide | K4D-24/6C59 |
| 21 | Peptide | K4D-20 |
| 22 | Peptide | K4A-12 |
| 23 | Peptide | K5D05 |
| 24 | Peptide | K5D-14 |
| 25 | Peptide | K4D-123 |
| 26 | Peptide | K4D-127/6C07 |
| 27 | Peptide | 5C14 |
| 28 | Peptide | 5C26 |
| 29 | Peptide | 5C27 |
| 30 | Peptide | 5C60 |
| 31 | Peptide | 6C13 |
| 32 | Peptide | 6C18 |
| 33 | Peptide | 6C54 |
| 34 | Peptide | 3C23K |
| 35 | Peptide | L-K55E |
| 36 | Peptide | L-T48I, L-P50S |
| 37 | Peptide | LT48I, L-K55E |
| 38 | Peptide | LS27P, L-S28P |
| 39 | Peptide | L-M4L, L-T20A |
| 40 | Peptide | L-S27P |
| 41 | Peptide | L-M4L, L-S9P, L-R31W |
| 42 | Peptide | L-M4L |
| 43 | Peptide | L-I33T |
| 44 | Peptide | L-M4L, L-K39E |
| 45 | Peptide | L-T22P |
| 46 | Peptide | L-Y32D |
| 47 | Peptide | L-Q37H |
| 48 | Peptide | L-G97S |
| 49 | Peptide | L-S12P |
| 50 | Peptide | L-19A |
| 51 | Peptide | L-T72A |
| 52 | Peptide | L-R31W |
| 53 | Peptide | L-M4L, L-M39K |
| 54 | Peptide | L-I2N |
| 55 | Peptide | L-G63C, L-W91C |
| 56 | Peptide | L-R31G |
| 57 | Peptide | L-I75F |
| 58 | Peptide | L-I2T |
| 59 | Peptide | L-I2T, L-K42R |
| 60 | Peptide | L-Y49H |
| 61 | Peptide | L-M4L, L-T20S, L-K39E |
| 62 | Peptide | L-T69P |
| 63 | Peptide | 9F7F11_VH |
| 64 | Peptide | 9F7F11_VL |
| 65 | Peptide | H4B121_VH |
| 66 | Peptide | H4B121_VL |
| 67 | Peptide | HE4B33_VH |
| 68 | Peptide | HE4B33_VL |
| 69 | Nucleic acid | Fc fragment IGHG1 coding sequence |
| 70 | Peptide | Fc fragment IGHG1 |
| 71 | Peptide | CDRL-1 |
| 72 | Peptide | CDRL-2 |
| 73 | Peptide | CDRL-3 |
| 74 | Peptide | CDRH-1 |
| 75 | Peptide | CDRH-2 |
| 76 | Peptide | CDRH-3 |

1. A method of inhibiting cancer-associated immunosuppression in a subject in need thereof, comprising
administering to the subject a therapeutically effective amount of a glyco-engineered Fc fragment-bearing compound.

2. The method according to claim 1, wherein the glyco-engineered Fc fragment-bearing compound is a hypofucosylated Fc fragment-bearing compound.

3. The method according to claim 1, wherein the glyco-engineered Fc fragment-bearing compound has two amino acid chains of SEQ ID NO. 70.

4. The according claim 1, wherein the glyco-engineered Fc fragment-bearing compound is a glyco-engineered antibody.

5. The method according to claim 4, wherein the glyco-engineered antibody is directed against a tumor antigen.

6. The method according to claim 5, wherein the tumor antigen is selected from the group consisting of HER2, HER3, HER4, and AMHRII.

7. The method according to claim 4, wherein the glyco-engineered antibody is selected from the group consisting of:
(i) a glyco-engineered 3C23K anti-AMHRII antibody comprising:
a) a light chain comprising SEQ ID NO: 2 and a heavy chain comprising SEQ ID NO: 4;
b) a light chain comprising SEQ ID NO: 6 and a heavy chain comprising SEQ ID NO: 8;
c) a light chain comprising SEQ ID NO: 10 and a heavy chain comprising SEQ ID NO: 12;
d) a light chain comprising SEQ ID NO: 14 and a heavy chain comprising SEQ ID NO: 16;
(ii) an anti-HER3 9F7F11 glyco-engineered antibody comprising (i) a heavy chain variable region of SEQ ID NO. 63 and (ii) a light chain variable region of SEQ ID NO. 64,
(iii) an anti-HER3 H4B121 glyco-engineered antibody comprising (i) a heavy chain variable region of SEQ ID NO. 65 and (ii) a light chain variable region of SEQ ID NO. 66, and
(iv) an anti-HER4 HE4B33 glyco-engineered antibody comprising (i) a heavy chain variable region of SEQ ID NO. 67 and (ii) a light chain variable region of SEQ ID NO. 68.

8. The method according to claim 1, wherein the method further comprises administering to the subject an inhibitory immune checkpoint protein inhibitor.

9. The method according to claim 8, wherein the inhibitory immune checkpoint protein inhibitor is selected from the group consisting of inhibitors of PD-1, PD-L1, PD-L2, BTLA, CTLA-4, A2AR, B7-H3 (CD276), B7-H4 (VTCN1), IDO, KIR, LAG3, TIM-3 and VISTA.

10. The method according to claim 9, wherein the inhibitory immune checkpoint protein inhibitor is an antibody directed against the inhibitory immune checkpoint protein, or an antigen-binding fragment thereof.

11. A pharmaceutical composition comprising (i) the glyco-engineered Fc fragment-bearing compound according to claim 1 and (ii) an inhibitory immune checkpoint protein inhibitor.

12. The pharmaceutical composition according to claim 11, wherein the glyco-engineered Fc fragment-bearing compound is a glyco-engineered antibody directed against a tumor antigen.

13. The pharmaceutical composition according to claim 11, wherein the inhibitory immune checkpoint protein inhibitor is selected from the group consisting of inhibitors of PD-1, PD-L1, PD-L2, BTLA, CTLA-4, A2AR, B7-H3 (CD276), B7-H4 (VTCN1), IDO, KIR, LAG3, TIM-3 and VISTA.

14. The pharmaceutical composition according to claim 11, wherein the inhibitory immune checkpoint protein inhibitor is an antibody directed against the inhibitory immune checkpoint protein, or an antigen-binding fragment thereof.


SEQUENCE LISTING (76 sequences)

SEQ ID NO: 1 - DNA, 318 bp, Artificial Sequence
  Synthetic 3C_23 VL without leader
  Synthetic 3C_23 VL without leader
  CDS 1..318
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctggaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaag

SEQ ID NO: 2 - protein, 106 aa, Artificial Sequence
  Synthetic [CDS]1..318 from SEQ ID NO 1
  Synthetic Construct [CDS]1..318 from SEQ ID NO 1
DIQMTQSPSTLSASVGDRVTITCRASSSVRYIAWYQQKPGKAPKLLTYPTSSLESGVPSRFSGSGSGTEFTLTISSLQPDDFATYYCLQWSSYPWTFGGGTKVEIK

SEQ ID NO: 3 - DNA, 345 bp, Artificial Sequence
  Synthetic 3C_23 VH without leader
  Synthetic 3C_23 VH without leader
  CDS 1..345
caggtgcggctggtgcagagcggggccgaggtgaagaagcctggagcctcagtgaaggtgagttgcaaggcctccggttacaccttcaccagctaccacatccactgggtcagacaggctcccggccagagactggagtggatgggctggatctaccctggagatgactccaccaagtactcccagaagttccagggtcgcgtgaccattaccagggacaccagcgcctccactgcctacatggagctgtcttccctgagatctgaggataccgcagtctactactgtacacggggggaccgctttgcttactgggggcagggcactctggtgaccgtctcgagc

SEQ ID NO: 4 - protein, 115 aa, Artificial Sequence
  Synthetic [CDS]1..345 from SEQ ID NO 3
  Synthetic Construct [CDS]1..345 from SEQ ID NO 3
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 5 - DNA, 318 bp, Artificial Sequence
  Synthetic 3C_23K VL without leader
  Synthetic 3C_23K VL without leader
  CDS 1..318
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctgaaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaag

SEQ ID NO: 6 - protein, 106 aa, Artificial Sequence
  Synthetic [CDS]1..318 from SEQ ID NO 5
  Synthetic Construct [CDS]1..318 from SEQ ID NO 5
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 7 - DNA, 345 bp, Artificial Sequence; identical to SEQ ID NO: 3
  Synthetic 3C_23K VH without leader
  Synthetic 3C_23K VH without leader
  CDS 1..345

SEQ ID NO: 8 - protein, 115 aa, Artificial Sequence; identical to SEQ ID NO: 4
  Synthetic [CDS]1..345 from SEQ ID NO 7
  Synthetic Construct [CDS]1..345 from SEQ ID NO 7

SEQ ID NO: 9 - DNA, 639 bp, Artificial Sequence
  Synthetic 3C_23 light chain without leader
  Synthetic 3C_23 light chain without leader
  CDS 1..639
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctggaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaagcggaccgtcgccgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - protein, 213 aa, Artificial Sequence
  Synthetic [CDS]1..639 from SEQ ID NO 9
  Synthetic Construct [CDS]1..639 from SEQ ID NO 9
DIQMTQSPSTLSASVGDRVTITCRASSSVRYIAWYQQKPGKAPKLLTYPTSSLESGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1335 bp, Artificial Sequence
  Synthetic 3C_23 heavy chain without leader
  Synthetic 3C_23 heavy chain without leader
  CDS 1..1335
caggtgcggctggtgcagagcggggccgaggtgaagaagcctggagcctcagtgaaggtgagttgcaaggcctccggttacaccttcaccagctaccacatccactgggtcagacaggctcccggccagagactggagtggatgggctggatctaccctggagatgactccaccaagtactcccagaagttccagggtcgcgtgaccattaccagggacaccagcgcctccactgcctacatggagctgtcttccctgagatctgaggataccgcagtctactactgtacacggggggaccgctttgcttactgggggcagggcactctggtgaccgtctcgagcgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 445 aa, Artificial Sequence
  Synthetic [CDS]1..1335 from SEQ ID NO 11
  Synthetic Construct [CDS]1..1335 from SEQ ID NO 11
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 639 bp, Artificial Sequence
  Synthetic 3C_23K light chain without leader
  Synthetic 3C_23K light chain without leader
  CDS 1..639
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctgaaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaagcggaccgtcgccgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 14 - protein, 213 aa, Artificial Sequence
  Synthetic [CDS]1..639 from SEQ ID NO 13
  Synthetic Construct [CDS]1..639 from SEQ ID NO 13
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 1335 bp, Artificial Sequence; identical to SEQ ID NO: 11
  Synthetic 3C_23K heavy chain without leader
  Synthetic 3C_23K heavy chain without leader
  CDS 1..1335

SEQ ID NO: 16 - protein, 445 aa, Artificial Sequence; identical to SEQ ID NO: 12
  Synthetic [CDS]1..1335 from SEQ ID NO 15
  Synthetic Construct [CDS]1..1335 from SEQ ID NO 15

SEQ ID NO: 17 - protein, 115 aa, Artificial Sequence; identical to SEQ ID NO: 4
  Synthetic 3C23K/3C23
  Synthetic 3C23K/3C23

SEQ ID NO: 18 - protein, 115 aa, Artificial Sequence
  Synthetic 3C23KR/6B78
  Synthetic 3C23KR/6B78
QVQLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 19 - protein, 115 aa, Artificial Sequence
  Synthetic 5B42
  Synthetic 5B42
QVQLVQSGAEVKKPGASVKVSCKASGYTFTSYHIHWVRQAPGQRLEWMGWIYPGDDSTKYSQKFQGRVTITRDASASTAYMELSSLRSEDTAVYYCTRGDRFAYWGQGTLVTVSS

SEQ ID NO: 20 - protein, 115 aa, Artificial Sequence
  Synthetic K4D-24/6C59
  Synthetic K4D-24/6C59
RVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 21 - protein, 115 aa, Artificial Sequence
  Synthetic K4D-20
  Synthetic K4D-20
QVRLVQSGAEVKKPGASVKVSCKASGYTFTSNHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 22 - protein, 115 aa, Artificial Sequence
  Synthetic K4A-12“
  Synthetic K4A-12
  Synthetic K4A-12
QVRLVQSGAEVKKPGTSVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 23 - protein, 115 aa, Artificial Sequence
  Synthetic K5D05”
  Synthetic K5D05
  Synthetic K5D05
QVRLVQSGAEVKKPGASVKVSCKASGYTFTGYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 24 - protein, 115 aa, Artificial Sequence
  Synthetic K5D-14“
  Synthetic K5D-14
  Synthetic K5D-14
QVRLVQSGAEVKKPGASVKVSCKASGYSFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 25 - protein, 115 aa, Artificial Sequence
  Synthetic K4D-123”
  Synthetic K4D-123
  Synthetic K4D-123
QVRLVQSGAEVKKPGASVKVSCKASGYTFTSYHIHWVRQAPGQSLEWMGWIYPGDDSTKYSQKFQGRVTITRDTSASTAYMELSSLRSEDTAVYYCTRGDRFAYWGQGTLVTVSS

SEQ ID NO: 26 - protein, 115 aa, Artificial Sequence
  Synthetic K4D-127/6C07“
  Synthetic K4D-127/6C07
  Synthetic K4D-127/6C07
QVRLVQSGAEVKKPGASVKVSCKASGYTFTSYHIHWVRQAPGQRLEWMGWIYPGDDSTKYSQKFQGRVTTTRDTSASTAYMELSSLRSEDTAVYYCTRGDRFAYWGQGTLVTVSS

SEQ ID NO: 27 - protein, 115 aa, Artificial Sequence
  Synthetic 5C14”
  Synthetic 5C14
  Synthetic 5C14
QVRLVQSGAEVKKPGASVKVSCKASGYTFTSYHIHWVRQAPGQRLEWMGWIYPGDDSTKYSQKFQGRVTFTRDTSASTAYMELSSLRSEDTAVYYCTRGDRFAYWGQGTLVTVSS

SEQ ID NO: 28 - protein, 115 aa, Artificial Sequence
  Synthetic 5C26“
  Synthetic 5C26
  Synthetic 5C26
QVRLVQSGAEVKKPGASVKVSCKASGYTFTSYHIHWVRQAPGQRLEWMGWIYPGDDSTKYSQKFQGRMTITRDTSASTAYMELSSLRSEDTAVYYCTRGDRFAYWGQGTLVTVSS

SEQ ID NO: 29 - protein, 115 aa, Artificial Sequence
  Synthetic 5C27”
  Synthetic 5C27
  Synthetic 5C27
QVRLVQSGAEVKKPGASVKVSCKASGYTFTSYHIHWVRQAPGQRPEWMGWIYPGDDSTKYSQKFQGRVTITRDTSASTAYMELSSLRSEDTAVYYCTRGDRFAYWGQGTLVTVSS

SEQ ID NO: 30 - protein, 115 aa, Artificial Sequence
  Synthetic 5C60“
  Synthetic 5C60
  Synthetic 5C60
QVRLVQSGAKVRKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 31 - protein, 115 aa, Artificial Sequence
  Synthetic 6C13”
  Synthetic 6C13
  6C13
QVRLVQSGAEVKKPGASVKVSCKASGYTFTSYHIHWVRQAPGQRLEWMGWIYPEDDSTKYSQKFQGRVTITRDTSASTAYMELSSLRSEDTAVYYCTRGDRFAYWGQGTLVTVSS

SEQ ID NO: 32 - protein, 115 aa, Artificial Sequence
  Synthetic 6C18“
  Synthetic 6C18
  6C18
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCARGDRFAYWGQGTLVTVSS

SEQ ID NO: 33 - protein, 115 aa, Artificial Sequence
  Synthetic 6C54”
  Synthetic 6C54
  Synthetic 6C54
QVRLVQSGAEVKKPGASVKVSCKASGYTFTSYHIHWVRQAPGQRLEWMGWIYPGDDSTKYSQKFQGRVTITRDTSASTAYMELSSLRPEDTAVYYCTRGDRFAYWGQGTLVTVSS

SEQ ID NO: 34 - protein, 106 aa, Artificial Sequence; identical to SEQ ID NO: 6
  Synthetic 3C23K“
  Synthetic 3C23K
  Synthetic 3C23K

SEQ ID NO: 35 - protein, 106 aa, Artificial Sequence; identical to SEQ ID NO: 2
  Synthetic L-K55E”
  Synthetic L-K55E
  Synthetic L-K55E

SEQ ID NO: 36 - protein, 106 aa, Artificial Sequence
  Synthetic L-T48I, L-P50S“
  Synthetic L-T48I, L-P50S
  Synthetic L-T48I, L-P50S
DIQMTQSPSTLSASVGDRVTITCRASSSVRYIAWYQQKPGKAPKLLIYSTSSLKSGVPSRFSGSGSGTEFTLTISSLQPDDFATYYCLQWSSYPWTFGGGTKVEIK

SEQ ID NO: 37 - protein, 106 aa, Artificial Sequence
  Synthetic LT48I, L-K55E”
  Synthetic LT48I, L-K55E
  Synthetic LT48I, L-K55E
DIQMTQSPSTLSASVGDRVTITCRASSSVRYIAWYQQKPGKAPKLLIYPTSSLESGVPSRFSGSGSGTEFTLTISSLQPDDFATYYCLQWSSYPWTFGGGTKVEIK

SEQ ID NO: 38 - protein, 106 aa, Artificial Sequence
  Synthetic LS27P, L-S28P“
  Synthetic LS27P, L-S28P
  Synthetic LS27P, L-S28P
DIQMTQSPSTLSASVGDRVTITCRASPPVRYIAWYQQKPGKAPKLLTYPTSSLKSGVPSRFSGSGSGTEFTLTISSLQPDDFATYYCLQWSSYPWTFGGGTKVEIK

SEQ ID NO: 39 - protein, 106 aa, Artificial Sequence
  Synthetic L-M4L, L-T20A”
  Synthetic L-M4L, L-T20A
  Synthetic L-M4L, L-T20A
DIQLTQSPSTLSASVGDRVAITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 40 - protein, 106 aa, Artificial Sequence
  Synthetic L-S27P“
  Synthetic L-S27P
  Synthetic L-S27P
DIQMTQSPSTLSASVGDRVTITCRASPSVRYIAWYQQKPGKAPKLLTYPTSSLKSGVPSRFSGSGSGTEFTLTISSLQPDDFATYYCLQWSSYPWTFGGGTKVEIK

SEQ ID NO: 41 - protein, 106 aa, Artificial Sequence
  Synthetic L-M4L, L-S9P, L-R31W”
  Synthetic L-M4L, L-S9P, L-R31W
  Synthetic L-M4L, L-S9P, L-R31W
DIQLTQSPPTLSASVGDRVTITCRASSSVWYIAWYQQKPGKAPKLLTYPTSSLKSGVPSRFSGSGSGTEFTLTISSLQPDDFATYYCLQWSSYPWTFGGGTKVEIK

SEQ ID NO: 42 - protein, 106 aa, Artificial Sequence
  Synthetic L-M4L“
  Synthetic L-M4L
  Synthetic L-M4L
DIQLTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 43 - protein, 106 aa, Artificial Sequence
  Synthetic L-I33T”
  Synthetic L-I33T
  Synthetic L-I33T
DIQMTQSPSTLSASVGDRVTITCRASSSVRYTAWYQQKPGKAPKLLTYPTSSLKSGVPSRFSGSGSGTEFTLTISSLQPDDFATYYCLQWSSYPWTFGGGTKVEIK

SEQ ID NO: 44 - protein, 106 aa, Artificial Sequence
  Synthetic L-M4L, L-K39E“
  Synthetic L-M4L, L-K39E
  Synthetic L-M4L, L-K39E
DIQLTQSPSTLSASVGDRVTITCRASSSVRYIAWYQQEPGKAPKLLTYPTSSLKSGVPSRFSGSGSGTEFTLTISSLQPDDFATYYCLQWSSYPWTFGGGTKVEIK

SEQ ID NO: 45 - protein, 106 aa, Artificial Sequence
  Synthetic L-T22P”
  Synthetic L-T22P
  Synthetic L-T22P
DIQMTQSPSTLSASVGDRVTIPCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 46 - protein, 106 aa, Artificial Sequence
  Synthetic L-Y32D“
  Synthetic L-Y32D
  Synthetic L-Y32D
DIQMTQSPSTLSASVGDRVTITCRASSSVRDIAWYQQKPGKAPKLLTYPTSSLKSGVPSRFSGSGSGTEFTLTISSLQPDDFATYYCLQWSSYPWTFGGGTKVEIK

SEQ ID NO: 47 - protein, 106 aa, Artificial Sequence
  Synthetic L-Q37H”
  Synthetic L-Q37H
  Synthetic L-Q37H
DIQMTQSPSTLSASVGDRVTITCRASSSVRYIAWYHQKPGKAPKLLTYPTSSLKSGVPSRFSGSGSGTEFTLTISSLQPDDFATYYCLQWSSYPWTFGGGTKVEIK

SEQ ID NO: 48 - protein, 106 aa, Artificial Sequence
  Synthetic L-G97S“
  Synthetic L-G97S
  Synthetic L-G97S
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFSGGTKVEIK

SEQ ID NO: 49 - protein, 106 aa, Artificial Sequence
  Synthetic L-S12P”
  Synthetic L-S12P
  Synthetic L-S12P
DIQMTQSPSTLPASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 50 - protein, 106 aa, Artificial Sequence
  Synthetic L-19A“
  Synthetic L-19A
  Synthetic L-19A
DIQMTQSPSTLSASVGDRATITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 51 - protein, 106 aa, Artificial Sequence
  Synthetic L-T72A”
  Synthetic L-T72A
  Synthetic L-T72A
DIQMTQSPSTLSASVGDRVTITCRASSSVRYIAWYQQKPGKAPKLLTYPTSSLKSGVPSRFSGSGSGTEFALTISSLQPDDFATYYCLQWSSYPWTFGGGTKVEIK

SEQ ID NO: 52 - protein, 106 aa, Artificial Sequence
  Synthetic L-R31W“
  Synthetic L-R31W
  Synthetic L-R31W
DIQMTQSPSTLSASVGDRVTITCRASSSVWYIAWYQQKPGKAPKLLTYPTSSLKSGVPSRFSGSGSGTEFTLTISSLQPDDFATYYCLQWSSYPWTFGGGTKVEIK

SEQ ID NO: 53 - protein, 106 aa, Artificial Sequence
  Synthetic L-M4L, L-M39K”
  Synthetic L-M4L, L-M39K
  Synthetic L-M4L, L-M39K
DIQLTQSPSTLSASVGDRVTITCRASSSVRYIAWYQQMPGKAPKLLTYPTSSLKSGVPSRFSGSGSGTEFTLTISSLQPDDFATYYCLQWSSYPWTFGGGTKVEIK

SEQ ID NO: 54 - protein, 106 aa, Artificial Sequence
  Synthetic L-I2N“
  Synthetic L-I2N
  Synthetic L-I2N
DNQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 55 - protein, 106 aa, Artificial Sequence
  Synthetic L-G63C, L-W91C”
  Synthetic L-G63C, L-W91C
  Synthetic L-G63C, L-W91C
DIQMTQSPSTLSASVGDRVTITCRASSSVRYIAWYQQKPGKAPKLLTYPTSSLKSGVPSRFSCSGSGTEFTLTISSLQPDDFATYYCLQCSSYPWTFGGGTKVEIK

SEQ ID NO: 56 - protein, 106 aa, Artificial Sequence
  Synthetic L-R31G“
  Synthetic L-R31G
  Synthetic L-R31G
DIQMTQSPSTLSASVGDRVTITCRASSSVGYIAWYQQKPGKAPKLLTYPTSSLKSGVPSRFSGSGSGTEFTLTISSLQPDDFATYYCLQWSSYPWTFGGGTKVEIK

SEQ ID NO: 57 - protein, 106 aa, Artificial Sequence
  Synthetic L-I75F”
  Synthetic L-I75F
  Synthetic L-I75F
DIQMTQSPSTLSASVGDRVTITCRASSSVRYIAWYQQKPGKAPKLLTYPTSSLKSGVPSRFSGSGSGTEFTLTFSSLQPDDFATYYCLQWSSYPWTFGGGTKVEIK

SEQ ID NO: 58 - protein, 106 aa, Artificial Sequence
  Synthetic L-I2T“
  Synthetic L-I2T
  Synthetic L-I2T
DTQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 59 - protein, 106 aa, Artificial Sequence
  Synthetic L-I2T, L-K42R”
  Synthetic L-I2T, L-K42R
  Synthetic L-I2T, L-K42R
DTQMTQSPSTLSASVGDRVTITCRASSSVRYIAWYQQKPGRAPKLLTYPTSSLKSGVPSRFSGSGSGTEFTLTISSLQPDDFATYYCLQWSSYPWTFGGGTKVEIK

SEQ ID NO: 60 - protein, 106 aa, Artificial Sequence
  Synthetic L-Y49H“
  Synthetic L-Y49H
  Synthetic L-Y49H
DIQMTQSPSTLSASVGDRVTITCRASSSVRYIAWYQQKPGKAPKLLTHPTSSLKSGVPSRFSGSGSGTEFTLTISSLQPDDFATYYCLQWSSYPWTFGGGTKVEIK

SEQ ID NO: 61 - protein, 106 aa, Artificial Sequence
  Synthetic L-M4L, L-T20S, L-K39E”
  Synthetic L-M4L, L-T20S, L-K39E
  Synthetic L-M4L, L-T20S, L-K39E
DIQLTQSPSTLSASVGDRVSITCRASSSVRYIAWYQQEPGKAPKLLTYPTSSLKSGVPSRFSGSGSGTEFTLTISSLQPDDFATYYCLQWSSYPWTFGGGTKVEIK

SEQ ID NO: 62 - protein, 106 aa, Artificial Sequence
  Synthetic L-T69P“
  Synthetic L-T69P
  Synthetic L-T69P
DIQMTQSPSTLSASVGDRVTITCRASSSVRYIAWYQQKPGKAPKLLTYPTSSLKSGVPSRFSGSGSGPEFTLTISSLQPDDFATYYCLQWSSYPWTFGGGTKVEIK

SEQ ID NO: 63 - protein, 117 aa, Artificial Sequence
  Synthetic 9F7F11_VH
  Synthetic 9F7F11_VH
EVKLVESGGGLVQPGGSLKLSCAASGFTFSSYTMSWVRQTPEKRLEWVAYISDGGGVTYYPDTIKGRFTISRDNAKNTLYLQMSSLKSEDTAMYYCARDRYGLFAYWGQGTLVTVSA

SEQ ID NO: 64 - protein, 107 aa, Artificial Sequence
  Synthetic 9F7F11_VL
  Synthetic 9F7F11_VL
DIVMTQSQKFMSTSVGDRVSITCKASQNVGIAVAWYQQKPGQSPKLLIYSASNRYTGVPDRFTGSGSGTDFTLTISNMQSEDLADYFCQQYSNYPYTFGGGTKLEIK

SEQ ID NO: 65 - protein, 120 aa, Artificial Sequence
  Synthetic H4B121_VH
  Synthetic H4B121_VH
EVQLVESGGGLVQPGRSLRLSCAASGFTFDDYAMHWVRQAPGKGLEWVSGISWNSGSIGYADSVKGRFTISRDNAKNSLYLQMNSLRAEDTAVYYCAREGQWPNYGMDVWGQGTTVTVSS

SEQ ID NO: 66 - protein, 110 aa, Artificial Sequence
  Synthetic H4B121_VL
  Synthetic H4B121_VL
QSVLTQPPSVSAAPGQKVTISCPGSSSNIGNNYVSWYQQLPGTAPKLLIYDNNERPSGIPDRFSGSTSGTSATLDITDLQAEDEATYYCGAWDNTLGVYVLGTGTQLTVL

SEQ ID NO: 67 - protein, 119 aa, Artificial Sequence
  Synthetic HE4B33_VH
  Synthetic HE4B33_VH
EVQLVQSGAEVKKPGASVKVSCKASGYTFTSYGISWVRQAPGQGLEWMGGIIPIFGTANYAQKFQGRVTITADTSTSTVYMELSSLRSEDTAVYFCARSRGYYDPFDVWGQGTLVTVSS

SEQ ID NO: 68 - protein, 110 aa, Artificial Sequence
  Synthetic HE4B33_VL
  Synthetic HE4B33_VL
QSVVTQPPSVSAAPGQKVTISCSGSSSNIGNNYVSWYQQLPGTAPKLLIYDNNKRPSGIPDRFSASKSGTSATLDITGLQTGDEAHYYCGAWDSSLSVAVFGGGTKLTVL

SEQ ID NO: 69 - DNA, 990 bp, Artificial Sequence
  Synthetic Fc fragment IGHG1
  Synthetic Fc fragment IGHG1
gccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 70 - protein, 330 aa, Artificial Sequence
  Synthetic Fc fragment IGHG1
  Synthetic Fc fragment IGHG1
  VARIANT 97: Xaa means K or R
  VARIANT 239: Xaa means D or E
  VARIANT 241: Xaa means L or M
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 10 aa, Artificial Sequence
  Synthetic CDRL-1 of anti-AMHRII antibodies
  VARIANT 4: Xaa in position 4 is S or P
  VARIANT 5: Xaa in position 5 is S or P
  VARIANT 7: Xaa in position 7 is R or W or G
  VARIANT 8: Xaa in position 8 is T or D
  VARIANT 9: Xaa in position 9 is I or T
RASXXVXXXA

SEQ ID NO: 72 - protein, 7 aa, Artificial Sequence
  Synthetic CDRL-2 of anti-AMHRII antibodies
  VARIANT 6: Xaa in position 6 is K or E
PTSSLXS

SEQ ID NO: 73 - protein, 9 aa, Artificial Sequence
  Synthetic CDRL-3 of anti-AMHRII antibodies
LQWSSYPWT

SEQ ID NO: 74 - protein, 13 aa, Artificial Sequence
  Synthetic CDRH-1 of anti-AMHRII antibodies
  VARIANT 6: Xaa in position 6 is S or T
  VARIANT 9: Xaa in position 9 is S or G
  VARIANT 10: Xaa in position 10 is Y or N
KASGYXFTXXHIH

SEQ ID NO: 75 - protein, 17 aa, Artificial Sequence
  Synthetic CDRH-2 of anti-AMHRII antibodies
  VARIANT 5: Xaa in position 5 is G or E
WIYPXDDSTKYSQKFQG

SEQ ID NO: 76 - protein, 6 aa, Artificial Sequence
  Synthetic CDRH-3 of anti-AMHRII antibodies
GDRFAY